(19) United States
(12) Patent Application Publication
(10) Pub. No.: US 2022/0331487 A1
(43) Pub. Date: Oct. 20, 2022

(54) POLYMER-PERMEATED GRAFTS AND METHODS OF MAKING AND USING THE SAME

(71) Applicant: BIOAESTHETICS CORPORATION, RESEARCH TRIANGLE PARK, NC (US)

(72) Inventors: Nicholas C. PASHOS, Durham, NC (US); William M. HEIM, Durham, NC (US); David GRAHAM, Hillsborough, NC (US)

(21) Appl. No.: 17/765,951

(22) PCT Filed: Oct. 2, 2020
(86) PCT No.: PCT/US2020/053965

§ 371 Date: Apr. 1, 2022

Related U.S. Application Data
(60) Provisional application No. 62/910,038, filed on Oct. 3, 2019.

(51) Int. Cl. A61L 27/36; A61L 27/38; A61L 27/48

(52) U.S. Cl. CPC A61L 27/3683; A61L 27/3604; A61L 27/38; A61L 27/48; A61L 2430/40

(57) ABSTRACT

This invention is directed to polymer-permeated grafts, including those which contain an active which provide for the controlled or sustained release thereof, and methods of making and using the same.

### RELATED APPLICATIONS

This application claims priority to U.S. Prov. Appl. No. 62/910,038, filed on Oct. 3, 2019, the contents of which is incorporated by reference in its entirety.

All patents, patent applications and publications cited herein are hereby incorporated by reference in their entirety.

### FIELD OF THE INVENTION

This invention is directed to biocompatible polymer-permeated grafts, which comprise one or more polymers or resins permeated or impregnated therein, which polymers or resins optionally comprise one or more actives encapsulated therein, methods of making, and the therapeutic use of such polymer-permeated grafts, e.g., for tissue replacement and/or for effecting tissue regeneration or therapy in subjects in need thereof.

### BACKGROUND OF THE INVENTION

Plastination is a technique which uses polymers or resins to preserve bodies, body parts, anatomical specimens and surgical specimens in a physical state approaching that of the living condition. Generally preserved tissues obtained using conventional plastination methods are only useful in non-clinical settings, e.g., the preserved tissues are often used for teaching purposes or histological examination, as they are bioincompatible. By contrast the present invention provides polymer-permeated grafts, which optionally comprise one or more actives encapsulated therein, and methods of making, which possess attributes rendering them amenable for use, e.g., for tissue replacement and/or for effecting tissue regeneration and/or tissue reconstruction and/or augmentation in subjects in need thereof, and optionally for delivering an active to a desired site, e.g., via controlled release.

### SUMMARY OF THE INVENTION

The present invention provides biocompatible polymer-permeated grafts, which optionally comprise one or more actives encapsulated therein, and methods for making the same, which are suitable for use in subjects in need thereof, e.g., human subjects who have lost tissue as the result of surgery, infection, cancer or other disease or condition associated with tissue loss, burn, traumatic tissue injury and the like. In exemplary embodiments, the polymer-permeated graft comprises a decellularized or substantially decellularized tissue which has been treated under conditions whereby the graft substantially retains the basic architecture and gross structure of the endogenous tissue, and further optionally further retains the endogenous microarchitecture of the endogenous tissue (prior to decellularization), which graft comprises endogenous and/or exogenously added cellular matrix molecules, e.g., fibrous proteins and glycosaminoglycans (GAGs) comprised therein which include by way of example proteoglycans, e.g., glycosaminoglycans (GAGs) such as heparan sulfate, multi-domain proteins such as collagen XVIII, chondroitin sulfate, keratan sulfate, non-proteoglycan polysaccharides such as Hyaluronic acid, hyaluronan, proteins such as collagens which may comprise different forms, i.e., Fibrillar (Type I, II, III, V, XI), Facit (Type IX, XII, XIV), Short chain (Type VIII, X), Basement membrane (Type IV), and others (Type VI, VII, XIII), Elastins, tropoelastin, cell adhesion proteins such as fibronectins, laminins, and combinations of any of the foregoing.

In preferred exemplary embodiments these polymer-permeated grafts, which substantially retain the architecture of the treated tissue prior to decellularization, will be free or substantially free of endogenous cells as well as other materials which may preclude or be deleterious to biocompatibility such as nucleic acids, immunogenic materials, pathogens (e.g., viruses) or other materials comprised in the tissue which may pose safety or efficacy concerns in clinical usage.

In exemplary embodiments these polymer-permeated grafts, which substantially retain the architecture of the treated tissue prior to decellularization, will be permeated with one or more polymers, e.g., such that the polymers are substantially uniformly distributed throughout the polymer-permeated graft wherein optionally the one or more polymers permeated throughout the graft comprise at least one active comprised therein, which at least one active optionally is delivered to a desired tissue via controlled release during usage, e.g., a site where tissue has been lost or compromised, e.g., as the result of injury, disease, surgery, infection, trauma, et al.

In other exemplary embodiments these polymer-permeated grafts, which substantially retain the architecture of the treated tissue prior to decellularization will be permeated with a polymer, e.g., whereby the polymer is substantially uniformly distributed throughout the polymer-permeated graft, wherein optionally the polymer comprises at least one active comprised therein which is delivered to a desired tissue, e.g., via controlled release, further optionally which may be repopulated with desired cells prior to or after engraftment into a subject in need thereof.

In some embodiments, the decellularized tissue used to obtain the inventive polymer-permeated grafts is substantially free of water or is free of water.

In some embodiments, the polymer-permeated graft comprises less than about 50% polymer by weight (which “polymer” may comprise one or more different polymers or co-polymers). In some embodiments, the polymer-permeated graft comprises more than 0% polymer by weight, more than about 0.1% polymer by weight, more than about 1% polymer by weight, more than about 5% polymer by weight, more than about 10% polymer by weight, more than about 20% polymer by weight, more than about 30% polymer by weight, more than about 40% polymer by weight, or more than about 50% polymer by weight.

In some exemplary embodiments, the polymer-permeated graft may comprise or be derived from dermal tissue and/or an epidermal tissue.

In some exemplary embodiments, the polymer-permeated graft may further comprise synthetic or polymeric mesh materials, e.g., a dense polyethylene mesh or other biocompatible mesh material which optionally has been impregnated with one or more cellular matrix proteins which may promote the adhesion of cells to the graft.

In other exemplary embodiments, the polymer-permeated graft may comprise or be derived from any of the following: tissue or organ, e.g., lung, liver, muscle, ligament, bone, a nipple, areola, a nipple attached to an areola, a lip, skin, a tendon, an aorta or other blood vessel, an amniotic membrane, bone or demineralized bone, or a combination of any of the foregoing.

In exemplary embodiments, the decellularized tissue or polymer-permeated graft produced using same will comprise at least one extracellular matrix molecule which is present in the endogenous tissue and/or which is exogenously added during or after formation of the polymer-permeated graft. Non-limiting examples thereof include laminin, elastin, fibronectin, collagen (e.g., a Type I collagen, a Type III collagen, a Type IV collagen, a Type VI collagen, or a combination thereof), or a combination of any of the foregoing.

In some embodiments, the decellularized tissue or polymer-permeated graft produced using same is substantially free of skin, fat and/or fibrous tissue. In other embodiments the decellularized tissue or polymer-permeated graft produced using same may comprise skin, fat and/or fibrous tissue, e.g., in grafts for use in lipodystrophy patients or other plastic surgery applications.

In some embodiments, the polymer-permeated graft comprises a natural polymer (such as alginate, elastin, silk fibroin, collagen, et al.) and/or a synthetic polymer (such as cyanoacrylate, medical grade silicone, etc.), which polymers optionally may be in the form of a mixed or pure hydrogel.

In some embodiments, the polymer-permeated graft comprises an ink, dye or other colorant by itself or a polymer therein comprises an ink, dye or other colorant resulting in a colored polymer, such as a polymer comprising melanin, a dye, inks, e.g. inks used in tattoos, vegetable dye, or any other biocompatible dye, and combinations thereof. Preferably the color in the polymer will be stable at the desired site (e.g., maintain color at the site in the presence of tissue constituents, light, temperature et al.)

In some embodiments, a polymer in the polymer-permeated graft may comprise a viable cell, a biocompatible polymer comprising at least one active, (e.g., an antibiotic, growth factor, antibody, hormone, drug, cytokine, chemotherapeutic, analgesic, anti-inflammatory, anti-rejection agent, et. al.), a biodegradable polymer (for example, chitosan, collagen, alginate, cyanoacrylate, dermabond), a non-biodegradable polymer (for example, silicon, ultra-high-molecular-weight polyethylene (“UHMWPE”), a polymer capable of cross-linking, or a combination of any of the foregoing.

In some embodiments, viable cells and/or metabolically active cells are introduced into polymer-permeated graft ex vivo or under conditions whereby they repopulate the tissue with the introduced cells or progeny thereof. These viable cells and/or metabolically active cells may comprise exogenous cells, autologous cells, allogenic cells or xenogenic cells which optionally may be genetically engineered to eliminate or provide for the expression of a desired gene or genes, e.g., using CRSPR-CAS gene editing. Non-limiting examples of cells which may be introduced into the present polymer-permeated grafts include stromal cells, fibroblasts, endothelial cells, progenitor cells, adult stem cells, organ-specific cells, tissue-specific cells, keratinocytes, melanocytes, nerve cells, osteoclasts, muscle cells, blood cells, immune cells, or a combination of any of the foregoing.

The present invention further provides methods of making such polymer-permeated grafts which optionally comprise one or more encapsulated actives, which are suitable for use, e.g., in human subjects in need thereof.

In exemplary embodiments, methods of making the inventive polymer-permeated grafts will comprise obtaining a decellularized tissue¹ or organ; optionally dehydrating the tissue or organ by removing substantially all of the water to provide a tissue or organ that is free of or substantially free of water optionally using one or more dehydrating solvents, e.g., ethanol or another alcohol; optionally, fixing the decellularized tissue by submerging or impregnating the decellularized tissue with a fixative, e.g., an aldehyde such as formaldehyde or glutaraldehyde, genipin, or combinations thereof for a period of time sufficient to fix the tissue; optionally replacing substantially all of the water within the tissue by submerging the decellularized tissue in a solution comprising one or more rehydrating solvents, e.g., as acetone or methyl chloride for a period of time and at a temperature sufficient to replace all or substantially all of the water within the tissue; optionally, removing all or substantially all of the fat within the tissue by submerging the decellularized tissue in a solvent for a period of time and at a temperature sufficient to remove substantially all lipids; permeating the tissue with a polymer by submerging the tissue in a composition comprising the polymer and subjecting the submerged tissue to vacuum for a period of time sufficient to permeate the tissue with the polymer; optionally, cross-linking the polymer permeated within the tissue; wherein the foregoing steps may be effected in the same or different order, the method may comprise other treatment steps, wherein preferably the resultant polymer-permeated tissue comprises the polymer substantially uniformly distributed in said tissue; and further wherein the resultant polymer-permeated graft is suitable for use, preferably for use in human recipients. ¹ A “tissue” in biology generally refers to an ensemble of similar cells and their extracellular matrix from the same origin that together carry out a specific function. In the present application we refer to the decellularized tissue before and after decellularization and polymer-permeation as a “tissue” because it possesses the basic structure of the tissue prior to decellularization and optionally may be repopulated with desired cells.

In some embodiments, a polymer which is distributed throughout the tissue may be cross-linked, e.g., by the addition of a chemical or light inducible crosslinking agent or a temperature inducible crosslinker, which cross-linker optionally can be admixed with the polymer or precursor thereof prior to permeating the decellularized tissue and/or matrix therewith. Alternatively or additionally, a polymer or resin comprised in the tissue may be crosslinked by mechanical means, e.g., by shaking, mixing or submerging in a suitable composition (e.g., silk can form crosslinks by shaking and alginate can be crosslinked by submerging it in a calcium or polycation solution).

In some exemplary embodiments a tissue comprising epidermal and/or dermal cells and potentially other cell types is decellularized under conditions wherein at least one matrix molecule is retained, e.g., an extracellular matrix molecule. Non-limiting examples thereof include laminin, elastin, fibronectin, collagen (such as a Type I collagen, a Type III collagen, a Type IV collagen, a Type VI collagen, or a combination thereof), and others identified supra, or a combination of any of the foregoing.

In some embodiments the viable cells and/or metabolically active cells are added to the decellularized tissue ex vivo and/or under conditions whereby the tissue becomes repopulated with the added viable cell and/or metabolically active cells or progeny thereof. The repopulating step may be effected at or about the same time as the polymer permeating step, and/or the repopulating step may be effected after the polymer permeating step. The viable cells and/or metabolically active cells used to repopulate the graft can comprise autologous cells, and/or may comprise exogenous cells, e.g., allogenic cells or xenogenic cells. Non-limiting examples of cell types which may be added to repopulate the graft include stromal cells, fibroblasts, endothelial cells, progenitor cells, stem cells, organ-specific cells, tissue-specific cells, keratinocytes, melanocytes, nerve cells, muscle cells, osteoclasts, which potentially may be genetically modified, or combinations thereof. Also, proteins such as growth factors, hormones and other moieties which promote cell growth and/or cell differentiation may be added to the graft.

In some exemplary embodiments, the tissue or organ which is decellularized is treated with one or more fixatives during or after decellularization, e.g., an aldehyde such as formaldehyde or glutaraldehyde, genipin, or a combination of two or more fixatives optionally selected from the foregoing.

In some exemplary embodiments, the tissue or organ which is decellularized is treated with one or more hydrating solvents such as acetone, xylene, alkanes (linear, branched or cyclic) (e.g., pentane, hexane, heptane, octane, nonane, decane, et al.), acetaldehyde, or other solvents with high vapor pressure and mixtures thereof which further may be admixed with other solvents.

In some exemplary embodiments, the tissue or organ which is decellularized is treated with one or more dehydrating solvents such as ethanol, or another alcohol, dichloromethane, methyl chloride, acetonitrile, tetrahydrofuran, or a combination of any of the foregoing which may be admixed with other solvents.

In some embodiments, the decellularized tissue or organ which optionally is in a lyophilized, dried or hydrated state is incubated in a solvent, e.g., acetone or another high vapor pressure solvent, e.g., in some embodiments optimally at about −15° C. to 25° C. for a period of time. However, incubation may also be effected at higher and lower temperatures. Alternatively the tissue or organ is initially dehydrated, e.g., by incubating in an alcohol and then incubated in an acetone or another high vapor pressure solvent, e.g., at about −15° C. to 25° C. or at a higher or lower temperature if desired.

In some embodiments, the polymer which is distributed within the tissue is cross-linked, i.e., by crosslinking polymer to polymer and/or by crosslinking a polymer to the ECM, e.g., by the use of one or more UV cross-linking agents, chemical cross-linking agents or other art-recognized means or materials for effecting the formation of covalent bonds between or within the polymers in the graft and their immediate insoluble environment. The selection of cross-linker or linkers depends upon the specific polymer or co-polymer which is impregnated and its monomers and functional groups. Cross-linkers useful for cross-linking polymers are well known and include by way of example homobifunctional crosslinking reagents such as disuccinimidyl suberate or DSS, disuccinimidyl tartrate or DST) and dithiobis succinimidyl propionate, or DSP, and sulfhydryl-to-sulfhydryl crosslinkers such as BMOE and DTME, heterobifunctional crosslinkers such as (m-Maleimidobenzoyl-N-hydroxysuccinimide ester), GMBS (N-γ-Maleimidobutyryloxysuccinimide ester), EMCS (N-(ε-Maleimidocaproyloxy) succinimide ester) and sulfo-EMCS (N-(ε-Maleimidocaproyloxy) sulfo succinimide ester), and photoreactive crosslinkers such as aryl-azides and diazirines, e.g., (N-((2-pyridyldithio)ethyl)-4-azidosalicylamide), ANB-NOS (N-5-Azido-2-nitrobenzyloxysuccinimide), Sulfo-SANPAH, NHS-ester diazirines or azipentanoates.

The invention further includes in vitro uses of the subject polymer-impregnated grafts as well as in vivo uses, e.g., the invention includes therapeutic or clinical methods wherein one or several polymer-impregnated or permeated grafts according to the invention is/are introduced or grafted into a subject in need thereof. In general such methods comprise producing one or more polymer-permeated or impregnated grafts according to the invention, which optionally comprise repopulated cells and/or optionally comprise one or more actives, and implanting the one or more polymer-permeated grafts to one or more sites on the subject, e.g., a site or sites where tissue has been lost, e.g., through disease, injury or surgery, e.g., a cancer surgery, under conditions whereby the polymer-permeated graft or grafts become stably engrafted in the subject at the desired site or sites.

In some embodiments, the polymer-permeated graft is repopulated with viable cells and/or metabolically active cells prior to or after engraftment. For example desired cells may be injected at the site and/or autologous cells comprised at the site of engraftment may repopulate the graft. The viable cells and/or metabolically active cells can comprise autologous cells, allogenic cells or xenogenic cells which optionally may be genetically engineered to provide for the expression and/or lack of expression of desired genes. Non-limiting examples of cell types which may be repopulated into the graft include stromal cells, fibroblasts, endothelial cells, progenitor cells, stem cells, organ-specific cells, tissue-specific cells, keratinocytes, melanocytes, muscle cells, osteoclasts, nerve cells, blood cells, immune cells or combinations thereof. As previously mentioned compounds which facilitate cell differentiation and/or cell growth may also be added to the graft such as hormones, growth factors, cytokines, et al.

Other objects and advantages of this invention will become readily apparent from the ensuing description.

### BRIEF DESCRIPTION OF THE FIGURES

FIG. 1 shows a schematic of a method of making a polymer-permeated graft. (NAC: nipple areola complex).

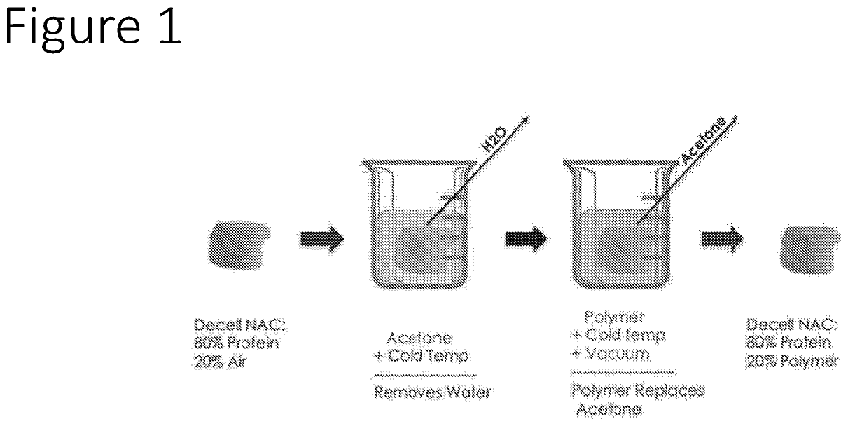

FIG. 2 shows a schematic of protein fibers and polymer structure.

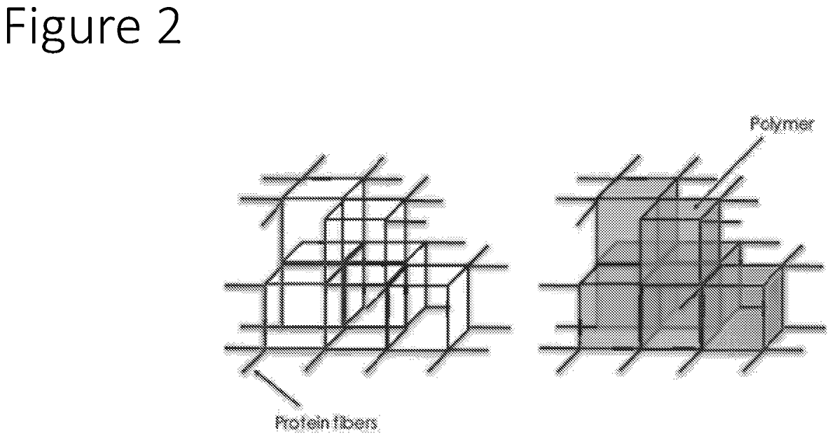

FIG. 3 provides a list of exemplary applications of a polymer-permeated graft.

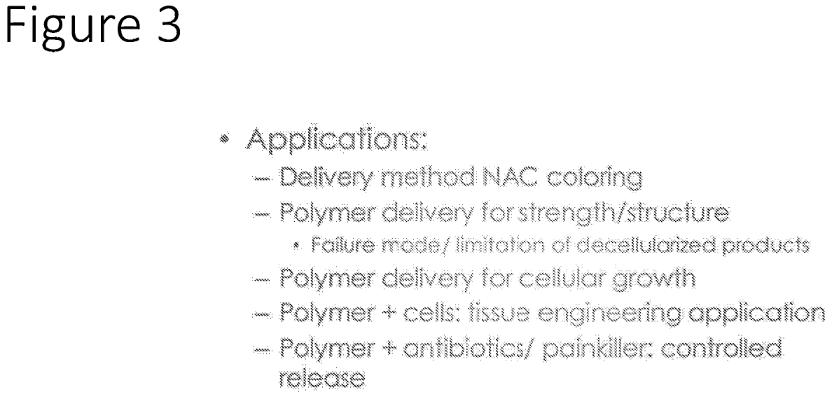

FIG. 4 shows electron microscope images of native (non-decellularized) and decellularized nipples (not polymer impregnated).

FIG. 5 is a schematic of the plastination process of the invention. (Top) An acetone soaked ABG is incubated in a polymer bath (purple) and subjected to vacuum force impregnation causing polymer to replace escaping acetone. ABG is washed and placed under bath conditions (polymer-dependent) to induce polymerization. (Bottom) Magnified view of ECM graft being vacuum force impregnated with monomers before crosslinking of monomers to polymers within interstitial space.

FIG. 6 is a schematic showing intact human skin that is decellularized and cut to size. Polymers are biotinylated and vacuum impregnated into acellular skin grafts using different combinations of polymer, concentration, and impregnation time. Impregnated polymers are then measured for polymer occupancy and distribution. Successful grafts are then measured for mechanical strength and bioactivity.

FIG. 7 shows histology of representative dcl-NHP NAC (murine model). (below) H&E micrograph of a randomly selected dcl-NHP NAC. Region in green box are shown in greater magnification in micrographs 1-3.

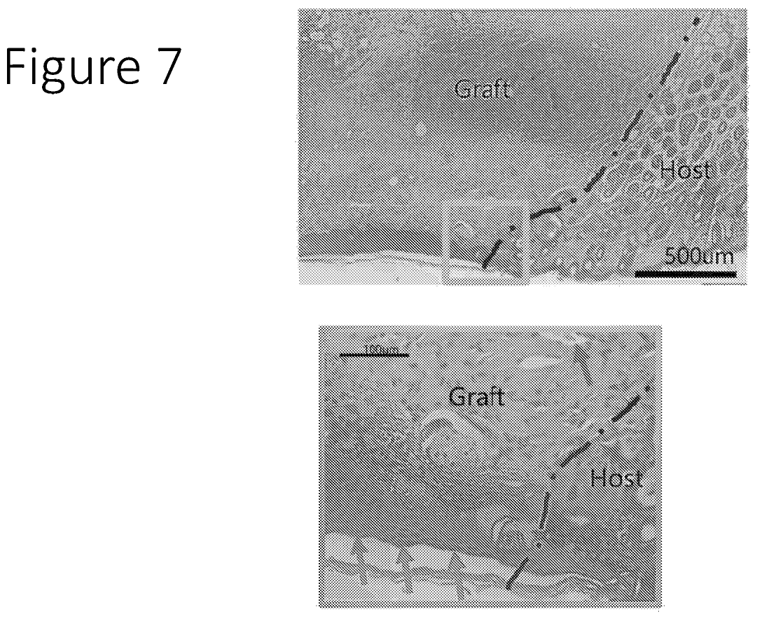

FIG. 8 is an illustration showing one process to generate drug loaded polyABG. Donor derived human skin is decellularized using our patent-pending process and cut to size. Grafts are saturated in an organic solvent, submerged in a polymer and drug bath mixture, and subject to vacuum pressure. The high vapor pressure of the organic solvent causes escape from the graft, creating a change in pressure within the interstitium. Upon return to normal pressure, the surrounding polymer and drug mixture is force-impregnated into the graft. The drug loaded polyABG is washed and placed under conditions to allow polymerization of polymer into a hydrogel which physically entraps the drug.

FIG. 9 is a table showing drug+polyABG embodiments described herein compared to current clinical products.

FIG. 10 shows data for dermal polyABGs. Dehydrated, acetone-saturated acellular human skin (1×1×0.4 cm) was incubated in the presence of gelatin or silk fibroin solutions under identical conditions, with only pressure varying. “Impregnation”: vacuum applied. “Diffusion”: atmospheric pressure. “Control”: dehydrated and acetone saturated acellular human skin in the presence of carrier solution (PBS). (Top) H&E stains of gelatin-impregnated acellular human skin. Gelatin appears as light pink stain within the interstitium. (Bottom) Alcian blue stains of silk fibroin-impregnated acellular human skin. Silk fibroin appears as dark blue stain within the interstitium. Images shown are from the center of the graft at a ˜2 mm depth from the graft surface. Hydrogel formation was induced by exposing polyABG to chilled PBS bath for gelatin and methanol for silk fibroin.

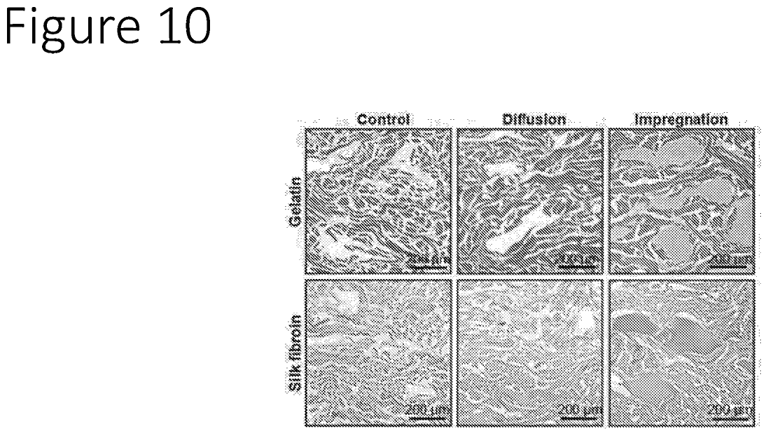

FIG. 11 shows cryo-scanning electron micrographs of intact and acellular skin from rhesus macaque showing collagen bundles and fibers in the epidermis and dermis.

FIG. 12: S10 plastinated acellular human dermis. (Top) Approximately 2 mm thick dermis was plastinated via the S10 plastination technique. Yellow box is magnified in bottom panel. (Bottom) Yellow arrows point to typical appearance of silicone. Note silicone's close association with collagen.

FIG. 13: S10 plastinated decellularized human NAC. (Top) Approximately 10 mm thick decellularized human NAC was plastinated via the S10 plastination technique. Yellow box is magnified in bottom panel. (Bottom) Though difficult to see, the grafts interstitia is replete with silicone.

FIG. 14: Dye impregnated acellular biologic graft. (Left) Control biologic graft impregnated with PBS. (Right) Approximately 2 mm thick acellular biologic graft (AlloMax®) impregnated with red dye via a patent-pending approach detailed below. Colored boxes show select magnified regions of tissue.

FIG. 15: Illustration of tissue impregnation. Intact donor skin is decellularized and cut to size. The tissue is chemically dehydrated and saturated in an organic solvent. The tissue is placed in a polymer bath inside a pressure vessel. The vessel is subjected to vacuum evaporating solvent from tissue. The vacuum is removed and vessel re-pressurized forcing dye into the tissue. Finally, the tissue is washed. The samples labeled “PBS”, “red dye” and “yellow dye” are ABGs impregnated via this method.

FIG. 16: Polymer impregnation of dermal acellular biologic graft with gelatin and silk fibroin. Dermal grafts (AlloMax®, 1×1×0.4 cm) were incubated in the presence of silk fibroin solutions under identical conditions with pressure varying. “Untreated” is in the presence of carrier solution (PBS). “Diffusion” is in the presence of silk fibroin but under atmospheric pressure. “Impregnation” is in the presence of silk fibroin but under varying vacuum conditions, resulting in a low percentage of impregnation (low) or high percentage of impregnation (high). (Top row) H&E stain showing gelatin impregnated graft. (Bottom row) Alcian blue stain showing silk fibroin impregnated graft.

FIG. 17: Silk fibroin impregnation of lyophilized versus acetone saturated dermal ABG. AlloMax was impregnated with either PBS or silk fibroin under identical conditions. AlloMax was either pre-processed and saturated in acetone or simply used in a lyophilized state.

FIG. 18. Silk fibroin impregnated polyABG (silkABG) has greater complex modulus than ABG alone. (Left images) Alcian blue stains of dermal ABGs from the same donor that were impregnated with PBS (ABG) or silk fibroin (silkABG). Increased blue staining in silkABG sample is of silk fibroin in graft interstitium. (Right) Graph showing rheological data measuring the complex modulus (elasticity and viscosity of material) for dermal ABGs from the same donor that were impregnated with PBS (ABG) or silk fibroin (silkABG). n=3 ABGs/treatment. Two-way ANOVA, simple effects performed. *p<0.001.

FIG. 19. Time-based release of rhodamine blue dye from silk fibroin impregnated polyABG DDS. Impregnation began with 50 μM rhodamine dye in silk monomer solution. Mixed solution was impregnated according to detailed protocol, actual rhodamine dye amount impregnated is not known. At end of experiment (14 days), the polyABG was releasing a 2.5 μM dye concentration.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein the term “acellular biologic graft” (ABG) refers to an acellular graft derived by decellularizing a natural or synthetic tissue or organ as described infra that comprises at least one matrix molecule from the by decellularized tissue or organ which preferably substantially retains the basic or gross structure or architecture of the tissue or organ prior to decellularization and further desirably retains some or all of the microarchitecture of the natural or synthetic tissue or organ prior to decellularization. This ABG optionally may be repopulated with desired cells before or after introduction into a recipient (as described in more detail infra). Also, the ABG optionally may comprise other moieties, e.g., drugs, growth factors, antibiotics, anti-rejection drugs, other moieties which promote stable engraftment, repopulation and growth of cells, prevent rejection, enhance stability, as described infra.

As used herein the term “Polymer-permeated acellular biologic graft” (PPG) or Polymer-permeated graft” (PPG) or “Polymer-permeated tissue or organ” or “Decellularized Polymer-Permeated Graft” or DPPG generally refers to an acellular graft derived by decellularizing a natural or synthetic tissue or organ as described infra that comprises at least one matrix molecule (e.g., extracellular matrix molecules such as elastin, elastic fibers, Type I collagen, Type III collagen, Type IV collagen, Type VI collagen, vitronectin, osteonectin, tenascin, hyaluronan, fibronectin, laminin, among others described herein). Generally, the PPG or DPPG is substantially free of water and is permeated with one or more polymers, which are typically distributed substantially uniformly in said tissue or organ. However, in some instances, some portions of the graft may comprise different polymers and/or amounts thereof, e.g., in order to mimic a desired tissue or organ. Preferably the decellularized tissue or organ retains the basic or gross structure or architecture of the tissue or organ prior to decellularization and further desirably retains some or all of the microarchitecture of the natural or synthetic tissue or organ prior to decellularization after it is permeabilized or permeated with) one or more polymers or co-polymers, e.g., polymers which are biocompatible and/or are suitable for in vivo use such as proteins, gelatins or collagens, and which further may desirably possess or mimic properties of a desired tissue or organ (e.g., elasticity, strength, et al.) and which optionally may comprise one or more actives encapsulated therein. Generally the polymer-permeated graft is biocompatible, i.e., is suitable for use in a subject in need thereof and is obtained by decellularizing a desired natural or synthetic tissue or organ as described infra. This PPG or DPPG optionally may be repopulated with desired cells before or after introduction into a recipient (as described in more detail infra). Also, the PPG or DPPG optionally may comprise other moieties, e.g., actives such as drugs, growth factors, antibiotics, analgesics, anti-rejection drugs, chemotactic factors, antibodies, and/or other moieties which promote stable engraftment, repopulation and growth of cells, prevent rejection, enhance stability, or which elicit a desired therapeutic effect as described infra when permeated and/or impregnated in the graft.

As used herein the term “extracellular matrix” herein refers to the complex network of macromolecules filling the extracellular space in a tissue, e.g., skin tissue, muscle, bone, tendon, a blood vessel such as an aorta, breast tissue such as a nipple or areola, penile tissue, bladder tissue, et al. The extracellular matrix is composed of glycosaminoglycans (GAGs), often covalently linked to protein forming the proteoglycans, and fibrous proteins, including collagen, elastin, fibronectin, and laminin. More particularly the extracellular matrix comprises fibrous proteins and glycosaminoglycans (GAGs) which include by way of example proteoglycans, e.g., glycosaminoglycans (GAGs) such as heparan sulfate, multi-domain proteins such as perlecan, agrin, and collagen XVIII, chondroitin sulfate, keratan sulfate, non-proteoglycan polysaccharides such as Hyaluronic acid, hyaluronan, proteins such as collagens which comprise different forms, i.e., Fibrillar (Type I, II, III, V, XI), Facit (Type IX, XII, XIV), Short chain (Type VIII, X), Basement membrane (Type IV), and others (Type VI, VII, XIII), Elastins, tropoelastin, cell adhesion proteins such as fibronectins, laminins, and combinations of any of the foregoing.

The term “plastination” generally refers to techniques which use polymers to preserve bodies, body parts, body tissues, anatomical specimens and surgical specimens in a physical state approaching that of the living condition. In contrast to the invention, in conventional methods wherein tissues, body parts and the like are preserved by use of plastination, bioincompatible polymers or bioincompatible resins are used, and consequently the resultant preserved materials are not suitable for in vivo use. By contrast, in the present invention biocompatible polymers are introduced into tissues or organs and the resultant polymer impregnated tissues or organs are suitable for in vivo usage, e.g., in human or non-human recipients. During the plastination process, a curable polymer is pulled, or drawn into the structure of the tissue using a vacuum chamber such that the polymer becomes impregnated into the tissue and generally is crosslinked prior or after impregnation. Generally after plastination the polymers are stably impregnated throughout the tissues, optionally homogeneously, and they are not prone to deterioration in an environment.

As mentioned the subject DPPGs include compositions comprising decellularized polymer permeated grafts which can be implanted into a subject in need thereof, e.g., a human or non-human recipient. The decellularized materials used to produce such decellularized polymer permeated grafts may include autologous or allogenic human tissues (from living or dead (cadaver) sources), animal tissues, e.g. porcine, canine, feline, bovine, ovine, etc., and/or may comprise tissues or organs derived synthetically such as tissues derived ex vivo or in animals, e.g. derived from stem cells or synthetically created grafts that have cells grown on them, wherein such synthetically produced tissues or organs possess the desired architecture or gross structure and are later decellularized as described herein. Also, as described infra the DPPGs may include other constituents such as drugs, moieties which promote cell migration, cell repopulation, angiogenic factors, growth factors, antibiotics, anti-rejection agents, chemotherapeutics, antibodies, et al.

A “graft” herein can refer to the decellularized polymer permeated graft prior to use or alternatively may refer to a structure or composition comprising a polymer permeated or impregnated graft as above described, which optionally is repopulated with cells, prior to or after being implanted or attached to an individual to replace an anatomical feature or to correct an anatomical defect or it may be implanted for aesthetic reasons, e.g., in the case of tissue loss such tissue loss may be the result of injury, surgery, disease, birth abnormality, or in the case of aesthetic modification the graft may be used in breast, nose or chin augmentation, etc.

The grafts described herein, based on their desirable properties, can be used, such as replacing a tissue or an organ (for example lung, pancreas, kidney or liver), a muscle, a ligament, a bone, a nipple, areola, a nipple attached to an areola, a lip, skin, a tendon, an aorta or other blood vessel, an amniotic membrane, or a combination of any of the foregoing, e.g., at a site where tissue or organ has been surgically removed or has been lost due to disease, burn, trauma et al. Polymer-permeated grafts described herein can be made from any tissue, structure, or appendage of a subject's body or a synthetically derived tissue or organ as above-described that can be decellularized and/or substantially retains at least one matrix molecule after decellularization. Non-limiting examples thereof include skin or other tissues, organs (for example lung, pancreas, kidney or liver), a muscle, a ligament, a bone, a nipple, areola, a nipple attached to an areola, a lip, skin, a tendon, an aorta or other blood vessel, an amniotic membrane, or a combination of any of the foregoing. In exemplary embodiments, the graft comprises a dermal tissue and/or an epidermal tissue, such as a decellularized dermal tissue and/or epidermal tissue.

As noted the graft generally will comprise at least one matrix molecule, e.g., a molecule or component of the extracellular matrix and further may retain the gross structure of the tissue or organ after decellularization.

In the present invention the terms “extracellular matrix” or “ECM” herein generally refers to the complex network of macromolecules filling the extracellular space in a tissue, e.g., skin tissue, muscle, bone, tendon, a blood vessel such as an aorta, breast tissue such as a nipple or areola, penile tissue, bladder tissue, et al. In the present invention the ECM may be that of a natural tissue or organ or may be that of a synthetic tissue or organ, e.g., one produced ex vivo or (e.g., in a non-human animal such as a porcine) using stem cells and/or scaffolds which comprises different cells and which optionally possesses the gross structure or architecture of a desired tissue or organ. The extracellular matrix is composed of glycosaminoglycans (GAGs), often covalently linked to protein forming the proteoglycans, and fibrous proteins, including collagen, elastin, fibronectin, and laminin. For example the skin has a relatively high content of elastin and elastic fibers. Other extracellular matrix proteins include by way of example Type I collagen, Type III collagen, Type IV collagen, Type VI collagen, vitronectin, osteonectin, tenascin, and hyaluronan.

In the present invention the terms “extracellular matrix fibrous protein” or “cell adhesion molecule” includes any fibrous protein present in of the extracellular matrix, such as fibronectin, laminin, elastin or a collagen, including those described herein.

The graft can comprise a tissue that is cell free or is substantially free of cells. “Substantially free of cells” can describe a tissue or graft that has had at least 50%, 60%, 70%, 75%, 80%, 85%, 90%, 95%, 96%, 97%, 98%, 99% or more of the cells present in the tissue or structure removed. The percentage reduction in the number of cells compared to the tissue prior to decellularization can be determined by, for example, counting by visual inspection the number of cells visible in samples pre- and post-decellularization, along with DAPI staining to visualize nuclei. In some embodiments, the graft itself can be free of tissue or substantially free of tissue. For example, “substantially free of tissue” can describe a graft that has had at least 50%, 60%, 70%, 75%, 80%, 85%, 90%, 95%, 96%, 97%, 98%, 99% or more of the tissue or structure removed. In some embodiments, the graft itself can be free of cells or substantially free of cells (e.g., where the graft consists essentially of the extracellular matrix with or without the presence of polymer). For example, “a graft substantially free of cells” can describe a graft where at least 50%, 60%, 70%, 75%, 80%, 85%, 90%, 95%, 96%, 97%, 98%, 99% or more of the cells are absent from the graft structure (e.g., where the graft consists essentially of the extracellular matrix with or without the presence of polymer). In some embodiments, the graft can comprise essentially the extracellular matrix after a tissue has been decellularized. In some embodiments, the graft can comprise essentially the extracellular matrix after a tissue has been decellularized in addition to the presence of polymer.

The graft can comprise a tissue that is free of water (i.e., an anhydrous graft) or one that is substantially free of water. “Substantially free of water” can describe a tissue that has had at least 50%, 60%, 70%, 75%, 80%, 85%, 90%, 95%, 96%, 97%, 98%, 99% or more by weight of the water present in the tissue or structure removed.

The graft can also comprise a tissue that is free of skin, fat, lipids, and/or fibrous tissue. For example, “substantially free of skin, fat, lipids or fibrous tissue” can describe a tissue that has had at least about 50%, about 60%, about 70%, about 75%, about 80%, about 85%, about 90%, about 95%, about 96%, about 97%, about 98%, about 99% or more by weight of the skin, fat, lipids, or fibrous tissue present in the tissue or structure removed.

As mentioned the invention introduces one or more polymers such that they permeate into tissues and/or are forcefully impregnated into the tissues to make them stable and free from deterioration. In exemplary embodiments the polymers will comprise the following properties: easy and non-toxic to handle; a low viscosity in the uncured state; a base and a catalyst/resin activator mixture, optionally in a liquid phase that permits sufficient impregnation or permeation of the polymer throughout the tissue over time; the curing or crosslinking of the polymer is not inhibited or degraded by tissue constituents such as enzymes or other moieties in the graft; the polymer possesses appropriate mechanical properties after curing which will vary dependent upon the nature of the tissue, e.g., firmness, elasticity, and ideally will replicate or mimic those of the endogenous tissue or organ, biocompatibility, ability to be repopulated by cells, et al.

In some embodiments, the graft can comprise about 50% or more by weight of one or more polymers. In the context of the invention unless stated otherwise the percent of polymer will refer to the percentage by weight of the polymer in the graft. For example, the graft can comprise about 0.1% polymer to about 50% polymer by weight. Alternatively, the graft can comprise about 0.1% polymer and about 99.9% extracellular matrix by weight, or the graft can comprise about 1% polymer and about 99% extracellular matrix by weight, or about 50% polymer and about 50% extracellular matrix by weight. In particular embodiments, the graft can comprise about 10% to about 30% polymer by weight. In embodiments, the graft comprises about 10%, about 15%, about 20%, about 25%, or about 30% polymer by weight. In some exemplary embodiments, one or more polymers are substantially uniformly distributed in the graft. Also, in some exemplary embodiments, one or more actives are comprised within one or more polymers optionally substantially uniformly distributed in the graft, e.g., whereby the one or more actives are released at the engraftment site during in vivo usage.

In some embodiments, the one or more polymers can be provided in a solution that comprises about 50% by weight polymer or less. For example, the solution can comprise about 0.1% by weight of polymer to about 50% polymer by weight. For example, if the decellularized tissue is submerged in a bath comprising 1% polymer by weight, generally the amount of polymer within the newly formed “graft” would be less than 1%.

The polymer impregnated in the graft can be any biocompatible polymer which possesses desired stability and/or other desired properties, e.g., those that mimic that possessed by a desired specific tissue. For example such polymers can be a natural polymer, recombinant polymer, or a synthetic polymer or protein which may be isolated from a natural source such as plant or animal cells, grain, microbia, made recombinantly in an animal or desired cell, made chemically, et al. Natural polymers occur in nature and can be extracted, such as polysaccharides or proteins or natural polymers may also be made synthetically or recombinantly. Non-limiting examples of polysaccharides comprise chondroitin sulfate, heparin, heparan, alginic acid (i.e., alginate), hyaluronic acid, dermatan, dermatan sulfate, pectin, carboxymethyl cellulose, chitosan, melanin (and its derivatives, such as eumelanin, pheomelanin, and neuromelanin), agar, agarose, gellan, gum, and the like as well as their salt forms (such as sodium salt and potassium salt). Non-limiting examples of proteins include collagen, alkaline gelatin, acidic gelatin, gene recombination gelatin, among others identified herein and generally known in the art which proteins may be produced chemically or isolated and/or made recombinantly from plant or animal cells, grain, microbia or other sources, and the like.

Synthetic polymers are man-made molecules formed by the polymerization of desired monomers, e.g., macromolecules comprising polyacrylic acid, polyaspartic acid, polytartaric acid, polyglutamic acid, polyfumaric acid, and so on as well as their salt forms (such as sodium salt and potassium salt). Non-limiting examples of synthetic polymers useful herein comprise cyanoacrylate.

In some embodiments, the polymer can be a hydrogel. “Hydrogel” can refer to a broad class of polymeric materials with a three-dimensional cross-linked network structure containing a great deal of water, with the state between a liquid and a solid without fluidity. Such hydrogels may be used to encapsulate one or more actives which are released at a desired site after engraftment.

In some embodiments the graft may comprise a discrete (separate) dye, ink (e.g., tattoo ink) or other colorant.

In other embodiments the polymer itself can be colored and/or is capable of being colored, such as by a dye, ink, pigment, tattoo ink or other colorant. In some embodiments, the polymer comprises melanin, a dye, or a combination thereof. Such polymers dyes are known to those skilled in the art. For example, reference may be made to the following publications: U.S. Pat. Nos. 5,032,670, 4,999,418, 5,106,942, 5,030,708, U.S. Pat. Nos. 5,102,980, 5,043,376, 5,104,913, 5,281,659, 5,194,463 Patent, U.S. Pat. No. 4,804,719, and International Publication No. WO-92/07913.

In some embodiments, the polymer can be a biodegradable polymer, which can refer to a polymeric material that degrades under aerobic and/or anaerobic conditions in the presence of bacteria, fungi, algae, or other microorganisms, and/or proteases or other enzymes. Non-limiting examples of biodegradable polymers comprise chitosan, collagen, alginate, cyanoacrylate, Dermabond® (2-octyl cyanoacrylate); among others. In other embodiments, the polymer can be a non-biodegradable polymer, for example, rubber, silicon, ultra-high-molecular-weight polyethylene, (UHMWPE), combinations thereof, etc., The polymer or polymers in the graft which become impregnated throughout the tissue or organ may be selected based on the desired properties of the tissue or organ that the graft will replace or is grafted to.

In still other embodiments, the polymer can be a polymer blend. The concentrations of the various components within the polymer blend will depend on a number of factors, including the desired physical and mechanical properties of the final blend, the performance criteria of articles to be manufactured from a particular blend, the processing equipment used to manufacture and convert the blend into the desired article of manufacture, the particular components within the blend and the desired properties of the tissue or DPPG into which it will be encapsulated.

In some embodiments, the graft can be seeded with viable cells and/or metabolically active cells so as to repopulate the polymer-permeated graft with the viable cells and/or metabolically active cells. The term “viable cell” or “metabolically active cell” herein can refer to a cell, e.g. autologous, allogenic, xenogenic, that is alive and capable of growth, proliferation, migration, and/or differentiation. Also it specifically includes cells and cell compositions such as dermagraft (http://www.dermagraft.com/safety/), i.e., cells which are intended to die off overtime or a fixed time. For example while viable the cells may not proliferate, migrate and/or be treated in a manner which results in a short lifespan such as irradiation or another cell treatment which impairs cell proliferation and/or migration and the like.

The decellularized tissues and/or graft can act as structural scaffolds by which these cells can migrate and readily repopulate. In some embodiments, after implantation of the graft into a recipient, cells from the native tissue (i.e., from the host subject) or cells which are infused into the subject at the site of the graft, migrate into the structural scaffolds created through the decellularization process and repopulate the polymer-permeated graft.

For example, the decellularized tissues and/or the polymer-permeated graft can be seeded and incubated with exogenous cells under conditions conducive to repopulating the decellularized tissue and/or graft with the exogenous cells or cells derived from the exogenous cells. In some embodiments, the exogenous cells can be autologous, homologous (e.g., allogenic), or heterologous. Herein, “autologous” includes a biological material (e.g., exogenous cells) that is introduced into the same individual from whom the material was collected or derived.

Herein, “homologous” can refer to a biological material (e.g., exogenous cells or proteins) collected or derived from a compatible donor that will be introduced into a different individual from which the material was collected or derived. By contrast, “heterologous” includes a biological material (e.g., exogenous cells) collected or derived from a compatible donor of a different species that will be introduced into an individual. Non-limiting examples of exogenous cells that can be seeded onto (and thus useful for repopulating the decellularized tissue and/or polymer-permeated graft) include keratinocytes, melanocytes, nerve cells, stromal cells, fibroblasts, endothelial cells, progenitor cells, stem cells, organ-specific cells, tissue-specific cells, or a combination thereof. In some embodiments, keratinocytes readily migrate and repopulate decellularized dermis and/or decellularized epidermis. In some embodiments, melanocytes readily migrate and repopulate the decellularized tissue and/or polymer-permeated graft. In some embodiments, nerve cells readily migrate and repopulate the decellularized tissue and/or polymer-permeated graft. In further embodiments, the nerve cells can be neurospheres or neuronal cells.

Herein “exogenous [cells]” includes cells that have been introduced (e.g., seeded) to recellularize or repopulate a decellularized tissue which did not originate in the tissue prior to decellularization. For example, if a nipple or other dermal tissue from a subject is decellularized and repopulated with keratinocytes, melanocytes, and/or nerve cells originating from a skin punch taken from the same subject, the keratinocytes, melanocytes, and/or nerve cells are exogenous if they did not originate from the nipple or same dermal tissue.

Conditions conducive to repopulate the graft are dependent upon the cells used and the donor, and can include temperature, the presence or absence of growth factors, the presence or absence of differentiation factors or migration factors, the polymer used to permeate the tissue, or the air content. In embodiments, the polymer-permeated graft is introduced or implanted onto a subject, and the subject's own cells migrate into the graft. In other embodiments, viable cells and/or metabolically active cells are introduced into the tissue or graft prior to or after implanting the graft onto the subject. As mentioned optionally these cells may be recombinant or genetically engineered.

One of skill in the art can seed exogenous cells onto the decellularized tissue and/or graft by placing the structures into culture medium containing dissociated, or dissociated and expanded, cells and allowing the cells to migrate into the decellularized tissue or graft and repopulate the structures. In some embodiments, cells can be injected into one or more places proximate to or in the decellularized tissue or graft, such as into the interior, to accelerate repopulation of the structures.

The viable cells and/or metabolically active cells can be cultured prior to reseeding of the tissue or graft. The culture medium used to grow and expand cells of interest can be serum-free and may or may not include the use of feeder cells. Suitable media specific for keratinocytes are known in the art and include (but are not limited to): Keratinocyte Growth Medium 2 (PromoCell™ GmbH, Heidelberg, Germany); Stemline™ keratinocyte basal medium (Sigma-Aldrich Corp., St. Louis, Mo.); defined, BPE-free medium supplement (K 3136) (Sigma-Aldrich Corp.), and ATCC's Dermal Cell Basal Medium (PCS-200-030) supplemented with Keratinocyte Growth Kit™ (PCS-200-040). Suitable media specific for melanocytes are known in the art and include (but are not limited to) growth medium comprising insulin, ascorbic acid, glutamine, epinephrine, and calcium chloride. See, for example, ATCC Melanocyte Growth Kit™ (ATCC-PCS-200-041). Suitable media specific for nerve cells are known in the art and include (but are not limited to) growth medium comprising DMEM, 10% FBS, supplemented with NGF and L-glutamine.

In some embodiments the polymer can be admixed with at least one antibiotic. “Antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the polymer can be admixed with a wide variety of antibiotics, such as penicillins, cephalosporins, macrolides, fluoroquinolones, sulfonamides, tetracyclines, aminoglycosides, and the like.

In some embodiments the ABGs are impregnated with biodegradable polymers, such as those described herein, that provide enhanced mechanical properties, e.g., enhanced strength, elasticity, etc., such that polymer impregnation enhances the mechanical properties of the graft, e.g., allowing it to be more durable or flexible than a non-impregnated graft, thereby preventing or inhibiting graft failure due to mechanical forces.

In some preferred embodiments, the ABG and/or the polymer-impregnated ABG can further comprise therapeutics and/or drugs, that provide for the retention of the therapeutics and/or drugs at the site and/or which provide for sustained or controlled release of such therapeutics and/or drugs from the site. Such agents can be used to prevent and/or treat progression and/or symptoms of disease (such as those diseases and symptoms described herein), and can also be used to prevent, treat, and or alleviate unwanted side effects of graft implantation. Non-limiting examples of unwanted side effects of ABG implantation or grafting include pain, nausea, infection, inflammation, scarring, rejection, growth or influx of undesired cells, and the like. Such unwanted side effects can be prevented, treated, or relieved through sustained, controlled, local release of drugs and/or therapeutic agents from the polymer or the ABG. For example, the addition of at least one antibiotic, at least one anti-inflammatory, and/or at least one analgesic and/or anesthetic could prevent infection, reduce local inflammation and decrease pain at the surgical and/or implantation site, thus, for example, providing symptomatic relief.

An ABG and/or polymers optionally can be mixed with one or more therapeutic and/or prophylactic agents which are retained or are released, optionally sustainedly released, at the implantation site of the graft. These additional agents optionally may be ionically or covalently bound to the ABG, e.g., via cleavable linkers. Non-limiting examples of such agents include antibiotics, analgesics, anti-inflammatories, anti-rejection agent, anti-angiogenesis agents, angiogenesis promoting agents, antibodies, growth factors, cytokines, chemotherapeutics, and combinations thereof. The selection of therapeutic and/or prophylactic agents will depend upon the specific tissue or organ, condition of the subject and the like.

“Antibiotic” can refer to an agent that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the scaffold can be coated with a wide variety of antibiotics, such as penicillins, cephalosporins, macrolides, fluoroquinolones, sulfonamides, tetracyclines, aminoglycosides, and the like.

“Analgesic” can refer to an agent that can provide relief from pain. An analgesic is any member of a group of drugs used to achieve analgesia, i.e., relief from pain. For example, the analgesic can be a pyrazolone derivative, such as (ampyrone, dipyrone, antipyrine, aminopyrine, and propyphenazone), aspirin, paracetamol, a non-steroidal anti-inflammatory (such as Ibuprofen, diclofenac sodium, or naproxen sodium), an opioid (such as codeine phosphate, tramadol hydrochloride, morphine sulphate, oxycodone), or any combination thereof. An anesthetic refers to any member of a group of drugs used to induce anesthesia—in other words, to result in a temporary loss of sensation or awareness of pain. Non-limiting examples of anesthetics comprise benzocaine, chloroprocaine, cocaine, cyclomethycaine, dimethocaine, larocaine, piperocaine, propoxycaine, procaine, novocaine, proparacaine, tetracaine, amethocaine, articaine, bupivacaine, cinchocaine, dibucaine, etidocaine, levobupivacaine, lidocaine, lignocaine, mepivacaine, prilocaine, ropivacaine, trimecaine.

An “anti-inflammatory” refers to a substance that treats or reduces the severity of inflammation and/or swelling. Non-limiting examples of anti-inflammatories comprise steroidal anti-inflammatories (such as corticosteroids) and non-steroidal anti-inflammatories (such as aspirin, celecoxib, diclofenac, diflunisal, ibuprofen, indomethacin, ketoprofen, ketorolac, nabumetone, naproxen, oxaprozin, piroxicam, salsalate, sulindac, tolmetin).

Sustained-release or intermittent release grafts have a common goal of improving treatment and/or symptomatic relief over that achieved by their non-controlled counterparts. The use of an optimally designed sustained-release preparation in medical treatment can be characterized by a minimum of drug substance being employed to cure, control, and/or provide relief of the condition in a minimum amount of time. For example, the sustained-release grafts can release an amount of a drug over the course of a few hours, e.g., 1, 2, 3 . . . -12 or 1-24 hours, or days, e.g., 1 day, 1 week, 2 weeks, 3 weeks, 4 weeks, 2 months, 3 months, 4 months, 5 months, 6 months, 7 months, 8 months, 9 months, 10 months, 11 months, 12 months, or longer. Advantages of sustained-release formulations include extended activity of the drug, reduced dosage frequency, and increased patient compliance. In addition, sustained-release formulations can be used to affect the time of onset of action or other characteristics, such as blood levels of the drug, deliver drug{s} to desired site(s) and can thus inhibit the occurrence of adverse side effects such as loss of the graft, infection, pain, and the like.

Most sustained-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug desirably is released at a rate that will replace the amount of drug being metabolized and excreted from the body. Sustained-release of an active ingredient can be stimulated by various conditions including, but not limited to, pH, temperature, enzymes, water, or other physiological conditions or compounds.

Generally, PPGs according to the invention will comprise at least one cross-linked or cross-linkable polymer. A cross-link, or cross-link, refers to a bond, such as covalent bonds or ionic bonds, that links one polymer chain to another. Cross-linking can promote a difference in the polymers' physical properties. For example, a liquid polymer or resin can be turned into a semi-solid (i.e., gel) or solid by cross-linking the chains together. The resulting modification of mechanical properties depends strongly on the cross-link density. Low cross-link densities decrease the viscosities of polymer melts. Intermediate cross-link densities transform gummy polymers into materials that have high elastomeric properties and potentially high strengths. Very high cross-link densities can cause materials to become very rigid or glassy, such as phenol-formaldehyde materials. The skilled artisan will recognize that the extent of cross-linking and the specificities of cross-linking will vary based on the polymers utilized, the cross-linking methods utilized, and the desired outcome.

Plastinized anhydrous grafts described herein can be stored more easily and for longer periods of time than non-plastinized grafts. The storage conditions can be dependent on the properties of the graft itself, such as the polymer and/or the tissue utilized. As examples, the plastinized grafts can be stored either hydrated, dehydrated, or partially hydrated. For example, a hydrated graft can be stored in glycerol, saline, water, or organ preservation solution (such as perfadex). The grafts can be stored at any temperature between about −80° C. and about 4° C. or may be stored in liquid nitrogen or at room temperature or warmer, e.g., if sterilized or lyophilized the storage temperature threshold may be higher. The grafts can be lyophilized.

### Methods of Decellularization

Aspects of the invention are directed towards polymer-permeated grafts that comprise a tissue free or substantially free of cells (i.e., decellularized). “Decellularization” of a biological tissue or structure can refer to removing most or all of the cells of the tissue or structure. See, for example, U.S. patent application Ser. No. 15/523,306, which is incorporated by reference herein in its entirety. As described infra if the tissue used for decellularization is dried or lyophilized the procedure can be modified by the elimination of the dehydration step, e.g., using a solvent such as ethanol.

A “decellularized” biological tissue or structure (such as the dermis or epidermis herein), for example, can refer to removing most or all of the cells of the tissue or structure under conditions whereby the extracellular matrix (ECM) is substantially preserved and/or cell adhesion molecules are substantially preserved. As is well known in the art the extracellular matrix is a complex network of macromolecules filling the extracellular space in a tissue, e.g., dermis and/or epidermis and/or other desired tissue). The extracellular matrix has three main components: (1) viscous proteoglycans (e.g., glycosaminoglycans (GAGs) covalently linked to proteins), such as hyaluronan, heparan sulfate, keratan sulfate, chondroitin sulfate, and dermatan sulfate; (2) insoluble collagen fibers (proteins that provide strength) and elastin (proteins that provide resilience); and (3) soluble, fibrous ECM proteins (including fibronectin, and laminin) that bind proteoglycans and collagen fibers to receptors on the cell surface. Herein an “extracellular matrix fibrous protein” and “matrix molecule” each can refer to a fibrous protein in the extracellular matrix, such as fibronectin, laminin, elastin or collagen. In some embodiments, the collagen can comprise a Type I collagen, a Type III collagen, a Type IV collagen, a Type VI collagen, or a combination thereof.

In producing ABGs or PPGs according to the invention generally a tissue can be removed from a patient (e.g., self or non-self), from a cadaver, or from a non-human animal such as a porcine, bovine, ovine, primate, etc., which optionally may be genetically engineered, e.g., to eliminate expression of an antigen, delete any viral DNAs in the host (e.g., by use of CRSPR-CAS) and the like. Such removed tissues/structures can be referred to as a “donor tissue” and can be decellularized while retaining their natural gross structures, microarchitecture, and matrix molecules, including collagen, fibronectin, elastin and glycosaminoglycans.

Aspects of the invention are further directed towards preparing and/or using polymer-permeated grafts that comprise a tissue that has been substantially decellularized. “Decellularizing substantially all” cells of a described tissue or structure means that at least 50%, 60%, 70%, 75%, 80%, 85%, 90%, 95%, 96%, 97%, 98%, 99% or more of the cells present in the tissue or structure have been removed. The percentage reduction in the number of cells can be determined by, for example, counting by visual inspection the number of cells visible in samples pre- and post-decellularization, along with DAPI staining to visualize nuclei. However, notwithstanding the foregoing, the invention is intended to embrace any means for deceullarizing a tissue or organ that results in a decellularized material which may be used to derive PPGs according to the invention.

Also, organs may be decellularized, e.g., using the disclosed methods or as exemplified by U.S. Pat. No. 8,470,520, the entirety of which is incorporated herein by reference. Exemplary protocols according to the invention for decellularizing lung and organs comprise the following steps: (i) contacting the lung or other organ with a dilute solution of a detergent or surfactant capable of permeating eukaryotic cell membranes and solubilizing membrane proteins and incubation at 4° C.; (ii) changing or replacing the detergent or surfactant regularly, e.g., each day; (iii) after a sufficient time which may vary on the organ, e.g., after 4 days, washing the samples with water for several hours; (iv) then contacting and incubating the samples with a 2% solution of a bile salt, sodium deoxycholate (“SDC”), for 4 days at 4° C., with the bile salt solution changed each day. The samples are then subjected to a second wash with water, incubated with DNase I for a sufficient time, e.g., about 2 hours, again at 4° C., washed again with water and then stored. The solutions are then introduced into the lung or other organ by perfusion.

This protocol has been used to successfully substantially totally decellularize organs such as the lung and may be used to decellularize other tissues and organs. However, when used to decellularize nipple epidermis in studies using Rhesus macaque nipples, the same protocol decellularized approximately 95% of the dermis cells, but only 5% of the epidermal cells.

AlloDerm® and Glyaderm®, two commercially available acellular dermal matrices, are used as tissue extenders or dermal replacement for wounds and burns. Neither contains acellular epidermis. It is believed that these materials are made by separating the epidermis from the dermis prior to decellularizing the dermis. Without wishing to be bound by theory, it is believed that this is in part because the epidermis is denser than the dermis and much harder to decellularize.

To solve this problem, we performed studies to find protocols that would succeed in decellularizing nipple epidermis. After considerable efforts, the inventors succeeded in developing modifications that successfully decellularized nipple epidermis, along with the accompanying dermis. Given our results with decellularizing nipple epidermis and dermis, these protocols should be suitable for decellularizing skin epidermis and dermis on other tissues. In some embodiments, the epidermis of the donor tissue is decellularized along with the dermis.

In particular, to decellularize nipples, including the epidermis, we modified the protocol as follows. First, the temperatures of the incubations and washes were raised—rather than conducting them at 4° C., they were conducted at room temperature. Second, rather than changing the solutions each day during the multiple-day incubations, the samples were left in the same solution throughout the incubation. Without wishing to be bound by theory, it was thought this would augment digestion of the cells by not removing any endogenous proteases present in the cells. Third, the times of the incubations were doubled. Fourth, the concentration of the bile salt was doubled, with the concentration of the bile salt raised from 2% to 4%. Fourth, for the lung, the organ was perfused. As nipples do not have vessels allowing ready perfusion, the samples were initially agitated on an orbital shaker set to a rotation speed of 85-125 rpm, which we thought would subject the nipple to solutions in a manner simulating perfusion. We found, however, that the nipple did not decellularize at low speed agitation, but did when the shaker speed was increased to 325 rpm. While none of these changes by themselves or any two or three together were sufficient to decellularize the nipple samples, the combination of all four succeeded.

As the original decellularization protocol worked on the lung, which has airways permitting essentially all the tissue of the organ to be contacted with the detergents and other reagents, but did not work when used on the nipple, the nipple appears to be particularly resistant to decellularization of the epidermis. Accordingly, it is reasonable to expect that the protocol developed for decellularizing the nipple should be suitable for decellularizing other body parts for which an acellular matrix might be useful.

Based on the studies undertaken on the nipple, an exemplary method by which a sample comprising dermal and/or epidermal cells may be decellularized to remove dermal and epidermal cells is contacted with a first detergent or surfactant solution for 48 hours to about 144 hours, more preferably about 72 to about 120 hours, still more preferably about 80 to about 110 hours, even more preferably for about 96 hours, where “about” means±2 hours, which detergent or surfactant can permeate eukaryotic cell membranes and solubilize membrane proteins. Suitable detergents include 4-(1,1,3,3-Tetramethylbutyl)phenyl-polyethylene glycol (Triton™ X-100), octylphenoxypolyethoxy-ethanol (IGEPAL® CA-630), CHAPS (3-[(3-Cholamidopropyl)dimethylammonio]-1-propanesulfonate), sodium dodecyl sulfate and polyethylene glycol, with the first three being more preferred. The sample is then washed with water, preferably for several hours, and then contacted with an appropriate soluble bile salt as a second detergent for a time period similar to those described above for use with the first detergent, above. Sigma-Aldrich Corp. (St. Louis, Mo.), for example, sells at least the following sodium salts of bile acids: sodium cholate, sodium deoxycholate, sodium glycocholate, sodium taurocholate, and sodium taurodeoxycholate. While all of these salts have sodium as the cation, other cations can be used to form a salt of a bile acid for use in the inventive methods so long as the resulting bile salt is soluble and decellularizes epidermis. Any particular bile salt can be readily tested to see whether it is suitable for its use in decellularizing epidermis by using it in place of SDC in the decellularization protocol set forth in the Examples and subjecting the resulting sample to histological or immunological examination, or both, to determine whether substantially all epidermal cells of the nipple have been removed. If it has, the bile salt is suitable for use in the protocol.

The sample is then again washed with water for a few hours, preferably about two, and then washed again with a saline solution for a few hours, preferably about two, where “about” means plus or minus 15 minutes. The samples are then incubated overnight with 5× streptomycin-penicillin-amphotericin B (sold by a number of suppliers at 100×, including Sigma Aldrich, Lonza Walkerville, Inc. (Walkerville, Md.), the American Type Culture Collection (“ATCC”, Manassas, Va.), and EMD Millipore (Billerica, Mass.)), water washed, treated with deoxyribonuclease I (“DNase I”) for several hours, preferably about two hours, and then stored in a phosphate buffered saline solution containing 5× streptomycin-penicillin-amphotericin B at 4° C. until use. Using an orbital shaker as an exemplar, the rotations per minute, or rpm, is set to between about 250 and about 400, more preferably about 275 to about 375, still more preferably about 300 to about 350 and most preferably about 325, with “about” in this case meaning 5 rpm on either side of the stated number. Orbital shakers are preferred because of their smooth continuous motion and uniform mixing. However, other shakers known in the art may be used, e.g., rocking, rolling, reciprocal, overhead, vibrating platform, and rotating shakers. Additionally, other devices, such as rotators, that allow uniform mixing of the contents of containers over time may also be used. In general, any shaker, rotator or similar device that provides mechanical agitation of a sample can be used so long as it provides uniform mixing without being so violent that the mixing action disrupts the physical integrity of the sample, such as a nipple or NAC, being decellularized, i.e., it should not substantially impair the gross structure and extracellular structure of the tissue being decellularized. A person of skill will be readily able to determine the appropriate speed setting for the particular shaker or rotator used by reference to the rpm setting for an orbital shaker, as set forth above.

The use of decellularized tissues as disclosed herein provides better “scaffolds” for reseeding and formation of a graft which is for use. For example a desired tissue comprising dermal and/or epidermal cells can be decellularized and then repopulated or recellularized so that it comprises the cell types that may have been present in the tissue before it underwent decellularization using exogenous cells.

In this instance the term “exogenous” refers to cells which are introduced to recellularize a decellularized tissue i.e., the cells did not originate in the decellularized tissue (but may be from the same recipient). By way of example, if a nipple from a patient is decellularized and repopulated with keratinocytes originating from a skin punch taken from the same patient, as used herein, the keratinocytes are still exogenous to the decellularized nipple because they did not originate from the nipple.

### Methods of Plastination

An important focus of the invention is directed towards methods of plastinizing tissues so as to provide a plastinized (i.e., polymer-permeated) graft suitable for use. Steps of the plastination process can comprise fixation, dehydration, forced impregnation in a vacuum, and hardening. See, for example, Prasad, Ganesh, et al. “Preservation of tissue by plastination: A Review.” Int. J. Adv. Health Sci 1.11 (2015): 27-31; and U.S. Pat. No. 4,278,701, each of which are incorporated by reference herein in their entireties.

### Fixation

In the present invention “fixation” generally refers to the treatment of a decellularized tissue using one or more materials (“fixatives”) which act to preserve the biological material, e.g., by crosslinking biological materials comprised therein, thereby preventing or inhibiting decomposition of the resultant “fixed” tissues. However, in some embodiments, tissue fixation may not be required, e.g., if the tissue is very stable and/or if the decellularized tissue which is permeated with a desired polymer and potentially other cells is used relatively soon after it is produced, e.g., within several hours. The fixation process generally comprises submerging, immersing, or perfusing the tissue with a fixative for a sufficient period of time to fix the tissue. The skilled artisan will recognize that the time required to fix the tissue can depend upon the rate of penetration of the fixative, the type of tissue, the size of the tissue, the concentration of the fixative and additives used, the type of fixative, temperature, tonicity, and the like. Non-limiting examples of fixatives that can be used in the methods described herein comprise aldehydes (such as formaldehyde or glutaraldehyde), genipin, or combinations of two or more fixatives. In some exemplary embodiments the fixative process can take about 3 to 4 hours, but can be shorter or longer as needed.

### Dehydration

Herein the term “dehydration” generally refers to a process by which all or substantially all of the water is removed from the decellularized tissue to provide a tissue that is free of or substantially free of water. “Substantially free of water” can describe a tissue that has had at least 50%, 60%, 70%, 75%, 80%, 85%, 90%, 95%, 96%, 97%, 98%, 99% or more by weight of water present in the tissue or structure removed. Generally, this is effected using a dehydrating organic solvent such as an alcohol, e.g., ethanol.

In embodiments herein, all or substantially all of the water within the decellularized tissue is removed and/or replaced with an organic solvent by submerging, immersing, or perfusing the tissue with the dehydrating organic solvent, e.g., ethanol, for a period of time and at a temperature sufficient to remove all or substantially all of the water within the tissue and later treated with a “rehydrating organic solvent”, e.g., which acts to replace all or substantially all of the water in the tissue.

In some embodiments, all or substantially all of the lipids and/or fat within the tissue can be removed and/or replaced with a suitable organic solvent by submerging, immersing, or perfusing the tissue with the suitable solvent for a period of time and at a temperature sufficient to remove and/or replace all or substantially all of the fat and lipid within the tissue.

As mentioned previously “dehydrating solvents” include ethanol and other alcohols. The dehydrating organic solvent will draw out all or substantially all of the water from the tissue. By contrast, “rehydrating solvents” include acetone or methyl chloride, acetone, xylene, dichloromethane (i.e., DCM or methylene chloride), and other organic solvents having high vapor pressure which act to replace all or substantially all of the water in the tissue.

The skilled artisan will recognize that a period of time and temperature sufficient to remove and/or replace water from a desired tissue using various dehydrating and hydrating organic solvents can depend upon the rate of penetration of the particular solvent, the type of tissue, the size of the tissue, the concentration of the solvent and any additives to be used, the type of solvent used, tonicity, and the like. In an exemplary embodiment, the decellularized tissue can be rehydrated by incubating in acetone at about −15° C. to 25° C. for a period of time as short as about 5 minutes to as long as about 10 days (e.g., dependent on the desired amount of rehydration). However, these times and conditions may vary if using different solvents, tissues and the like.

In some embodiments, water in a tissue can be removed and/or replaced by sequential immersion of the tissue in repeated changes of organic solvents until all or substantially all of the water within the tissue is removed or replaced. The same organic solvent can be used in each bath, or different solvents can be used in the baths, generally beginning with aqueous ethanol or another alcohol, and progressing gradually to anhydrous ethanol and/or acetone which may then be followed by other organic solvents which will vary depending on the suit processing conditions, e.g., polymers used and polymerization conditions.

### Forced Permeation

Replacement of the solvent by one or more polymers is a central step in plastination, and generally comprises submerging or immersing the decellularized tissue in a bath of liquid polymer for a period of time under vacuum conditions. As the solvent leaves the tissue, e.g., by vaporizing, the liquid polymer is drawn into the tissue during the removal of the vacuum pressure and thereby permeates and/or impregnates the tissue. Typically it is preferred that the polymer be substantially uniformly distributed within the tissue.

In some embodiments, the polymer can comprise those described herein, non-limiting examples of which comprise acrylic resins, epoxy resins, polyester resins, polyurethanes, and silicone resins. The selection of a desired polymer or polymers to be permeated and/or impregnated in a tissue and appropriate polymerization and crosslinking conditions can be determined by a skilled person.

The solvent-bearing tissue can be permeated and/or impregnated with a polymer or precursor thereof by submerging, immersing or perfusing the tissue with a fluid composition comprising the polymer or precursor thereof, and optionally other additives such as catalysts or hardeners, chemical cross-linking agents (such as glutaraldehyde, carbodiimide (1-ethyl-3-(3-dimethyl aminopropyl)-carbodiimide), epoxy compounds, six methylene diisocyanate, glycerin, alginate, genipin, ordihydroguaiaretic acid, proanthocyanidin, tannic acid, and epigallocatechin gallate), accelerators, plasticizers, and like conventional ingredients. Impregnation of the immersed tissue can be aided by evaporating or otherwise releasing the organic solvent from the tissue, such as by vacuum, as the solvent can be more volatile than any component of the polymer solution. For example, exposing the tissue immersed in the polymer to a vacuum can cause impregnation or permeation in a very short time if the solvent is volatile in the vacuum, and/or the precursor composition is not overly viscous. Compositions up to about 5000 cps have been used without difficulty, and even more viscous compositions may be employed for impregnation by alternating application of negative and positive pressure. By contrast, much lower viscosity is necessary for successful impregnation using perfusion methods.

For example, the tissue, immersed and/or submerged within a volatile solvent can be removed from the solvent and placed into the polymer solution and potentially other moieties discussed above. The solvent can have a high vapor pressure and a low boiling point (for example, acetone: +56° C., methylene chloride: +40° C.), while the polymer solution has a low vapor pressure and a high boiling point. Thus, on application of vacuum, the solvent is continuously extracted out of the specimen as gaseous bubbles. Upon degassing, the “synthetic resin” or polymer is drawn into the specimen.

The skilled artisan will recognize that the rate of extraction of solvent from the tissue can vary and can depend on different properties of the tissue to be impregnated and/or the solvent which is to be extracted. If desired the rate of extraction of the solvent can be visually monitored by observing as bubbles gently rise to the surface and burst. When no more bubbles appear, the process is complete.

To monitor the changes in vacuum, a vacuum gauge or an Hg column is used and/or a manometer can be used. In some embodiments impregnation or permeation is complete when the absolute pressure has stabilized to around 2-10 mm Hg, e.g., for a few days. During this step the change in size of bubbles in the tissue can be visualized, and comprise the gaseous state of the “rehydrating solvent”. In some embodiments, the tissue can be left in the impregnation bath at atmospheric pressure for a period of time, such as 24 hours, to allow the equilibration of pressure of the polymer in the specimen and in the impregnation bath. After this step, the tissue can be removed.

### Cross-Linking

After immersion in a polymer solution, the tissue can be removed from the polymer and excess polymer can be drained off or removed by wiping and the like before polymerization conditions (i.e., cross-linking conditions or curing conditions) are established. Alternatively, crosslinking can occur without the addition of other catalysts as some polymers exhibit time-based crosslinking based on proximity of reactive groups.

In some embodiments cross-links can be formed by chemical reactions that are initiated by gas, light, heat, pressure, change in pH, radiation, cross-linking agents, and the like. Crosslinks are useful for preventing degeneration of the structural integrity of the scaffold that remains after decellularization, enhancing mechanical strength and reducing calcification of the matrix. For example, mixing of an unpolymerized or partially polymerized monomer with cross-linking agents can result in a chemical reaction that forms cross-links (i.e., polymerizes). The skilled artisan will recognize that the extent of cross-linking and the specificities of cross-linking will vary based on the polymer and precursors utilized, the cross-linking methods utilized, the cross-linking agents utilized, and the desired outcome.

An ideal biomaterial crosslinking agent demonstrates little to no cytotoxicity and is low cost. There are many crosslinking agents for fixing ECM-derived scaffolds, which may be classified as (i) chemical crosslinking agents and (ii) natural crosslinking agents. Exemplary chemical crosslinking agents include glutaraldehyde (GA), carbodiimide (1-ethyl-3-(3-dimethyl aminopropyl)-carbodiimide (EDC)), epoxy compounds, six methylene diisocyanate, for example; and natural crosslinking agents include genipin (GP), nordihydroguaiaretic acid (NDGA), tannic acid, collagen, glycerin, alginate, procyanidins (PC) such as proanthocyanidin, among many others. See Ma, Bing, et al. “Crosslinking strategies for preparation of extracellular matrix-derived cardiovascular scaffolds.” Regenerative Biomaterials 1.1 (2014): 81-89, which is incorporated by reference here in its entirety.

In exemplary embodiments, the surface and mechanical properties of the finished product (i.e., the polymer permeated graft that is grafted in a host) will mimic those of endogenous tissue. For example, silicone rubber precursor compositions of low viscosity may simulate the softness of the endogenous tissue.

Cross-linking can depend upon the polymer or polymers utilized. For example, gas curing can be used for plastination using silicone resin. In this technique, the decisive cross-linking curing agent is applied in a gaseous form to the tissue. The silicone-impregnated tissue is kept in a closed chamber and are exposed to a gaseous hardener which, on evaporation from a stock solution, is continuously circulating in the atmosphere of the chamber. A small membrane pump helps in the evaporation and circulation of the gas, leading to faster curing.

As another example, the curing of an epoxy-impregnated tissue or polymerizing emulsion-impregnated tissue can use the tissue amines present within the tissue itself for curing. These amines are effective accelerators and together with anhydrides, they are sufficient to fully cure the tissues.

As yet another examples, polymerization can be induced by exposure to a radiation source, such as electron beam exposure, gamma-radiation, or UV light. For example, electron beam processing can be used to obtain the C type of cross-linked polyethylene (Davis, F. J., 2004. Polymer Chemistry A Practical Approach. New York: Oxford University Press). Other types of cross-linked polyethylene can be made by addition of peroxide during extruding (type A) or by addition of a cross-linking agent (e.g. vinylsilane) and a catalyst during extruding and then performing a post-extrusion curing.

Polymerization conditions are chosen to suit the specific polymer and/or precursor composition employed within limits set by the need to avoid decomposition of the tissue, and can include elevated temperatures, irradiation with ultraviolet light, the presence of initiators or catalysts, and other factors known in themselves. If atmospheric oxygen unduly inhibits the curing of polyester resin, the impregnated object can be immersed in anhydrous glycerol or held in a nitrogen atmosphere during curing. As an example, the polymerization of silicone rubber is not inhibited by oxygen in the atmosphere, but can be retarded as needed by maintaining a low temperature (below 32° F.) during impregnation. Their index of refraction (n=1.405) enhances the natural appearance of the impregnated tissue surface, and the resiliency of the cured silicone rubber simulates the softness of the fresh tissue to some reduced degree.

In embodiments, for example when polyester or epoxy resins are employed as the polymer, the temperature of the polymer fluid composition can be monitored to avoid polymerization and a sharp increase in viscosity before excess resin precursor is removed.

### Methods of Treatment

Aspects of the invention are directed towards methods of using a polymer-permeated graft (e.g., an acellular biologic graft (ABG) described herein) to treat a subject in need thereof. The ABGs according to the invention can be alternatives to synthetic mesh implants for use, e.g., in pelvic organ prolapse (POP) and abdominal wall repair, hernia repair. The ABGs according to the invention can also serve as alternatives to slings for use, e.g., in breast reconstruction. Further, the ABGs according to the invention can be alternatives to supplemental supports and/or coverings for tissue (for example, replacement of synthetic of biologically derived grafts) for use with gums, tendons, breast, burns, and the like. For example, the subject may be in need of nipple repair or replacement, breast reconstruction, hernia repair or replacement, blood vessel repair or replacement, muscle repair or replacement, repair or replacement of whole organs, tendon repair or replacement, cleft lip repair or replacement, or palate repair or replacement. In some embodiments, an ABG can be used to treat pelvic organ prolapse (POP) in a subject, for example, for use in a subject undergoing a POP surgery.

In embodiments, the method can comprise obtaining a plastinized graft such as a polymer-permeated graft as described herein and securing the graft to a prepared site on the patient. In some embodiments, the method further comprises allowing time for cells from the patient to integrate into the body part.

The term “treating” herein can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

The term “subject” or “patient” can refer to any organism to which aspects of the invention can be administered, e.g., for experimental, diagnostic, prophylactic, and/or therapeutic purposes. Typical subjects to which compositions of the present disclosure may be administered will be mammals, particularly primates, especially humans. For veterinary applications, a wide variety of subjects will be suitable, e.g., livestock such as cattle, sheep, goats, cows, horses, swine, dogs, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

As used herein, the phrase “changed as compared to a control” sample or subject is understood as having a level of an analyte or diagnostic or therapeutic indicator (e.g., marker) to be detected at a level that is statistically different than a sample from a normal, untreated, or abnormal state control sample. The diagnostic or therapeutic indicator can be assessment of the growth of the tissue grafted or observation for lack of graft rejection. Determination of statistical significance is within the ability of those skilled in the art, e.g., the number of standard deviations from the mean that constitute a positive or negative result.

In some embodiments, the polymer-permeated graft can be implanting onto a prepared site on or within a subject in need thereof; thereby grafting to a subject the polymer-permeated graft.

In some embodiments, the graft has been repopulated with viable cells and/or metabolically active cells as described herein.

In other embodiments, the decellularized tissue has been not been repopulated or has only been partially repopulated by cells (such as keratinocytes, melanocytes, nerve cells, or a combination thereof either that were seeded or that migrated from the native tissue) before grafting onto a subject in need thereof.

Cells from the prepared bed (such as keratinocytes, skin stem cells, melanocytes, nerve cells, and fibroblasts) can migrate into the polymer-permeated graft and repopulate it. In some embodiments, the migration of cells into the graft is facilitated by (a) placing the graft on the subject on the prepared bed and (b) coating the graft and the junction where the graft adjoins the subject's skin with a biocompatible substance. For example, the biocompatible substance (or occlusive coating) can be a tissue sealant, a tissue adhesive, tissue glue, or a surgical glue. For example, “biocompatible” refers to a material which is not toxic, not injurious or not inhibitory to mammalian cells, tissues, or organs with which it comes in contact. Furthermore, when the material is in use with respect to a graft does not induce an immunological or inflammatory response sufficient to be deleterious to the subject's health or to engraftment of the graft. Other biocompatible occlusive coatings that provide an air sealing barrier, such as Fibrin glues, can be used. A fibrin sealant, TISSEEL®, is commercially available, as are the sealants BIOGLUE® and DuraSeal®. A non-limiting example of a tissue sealant includes high viscosity 2-octyl cyanoacrylate (for example, sold commercially under the names DERMABOND® (Ethicon unit of Johnson & Johnson) and Sure+Close®II).

Once the polymer-permeated graft is grafted onto the subject, it can be covered with a biocompatible occlusive coating as described herein. The skilled artisan can readily obtain keratinocytes, melanocytes, nerve cells, or a combination thereof, from one or more skin punches (either from the same subject or from a compatible donor) according to methods and teachings known in the art. The keratinocytes, melanocytes, nerve cells, or a combination thereof, can be placed in a culture medium suitable for maintenance and stability, from which the cells can then permeate into the graft. In some embodiments, these cells can also be injected into the graft at one or more locations. Grafts of the invention can be maintained in a cell culture medium suitable for maintenance and expansion of keratinocytes (human or non-human); cell culture medium suitable for maintenance and expansion of melanocytes (human or non-human); cell culture medium suitable for maintenance and expansion of nerve cells (human or non-human). The culture medium used to grow and expand cells of interest can be serum-free and would not require the use of feeder cells.

### Kits

The compositions and grafts as described herein can also be provided in a kit. In one embodiment, the kit includes (a) a container that contains a composition or a graft as described herein, and optionally (b) informational material. In another embodiment, the kit comprises vials comprising (a) a fixative, an organic solvent, a polymer, a chemical linker, or any combination thereof, and (b) informational material. The informational material can be descriptive, instructional, marketing or other material that relates to the methods described herein and/or the use of the composition, the graft for therapeutic benefit, or solutions. In an embodiment, the kit also includes a biocompatible sealant for treating a subject in need thereof.

The informational material of the kits is not limited in its form. In one embodiment, the informational material can include information about production of the composition or the graft, components of the composition or the graft, date of expiration, batch or production site information, and so forth. In one embodiment, the informational material relates to methods of administering or affixing the composition or the graft, e.g., in a suitable form, or mode of administration, to treat a subject. The information can be provided in a variety of formats, include printed text, computer readable material, video recording, or audio recording, or information that provides a link or address to substantive material. In addition to a composition or graft as described herein, the composition in the kit can include other ingredients, such as a buffer, a stabilizer, or a preservative. The composition or graft can be provided in a sterile form and prepackaged.

The kit can include one or more containers for the composition or grafts described herein. In some embodiments, the kit contains separate containers, dividers or compartments for the composition or graft and informational material. For example, the composition can be contained in a culture plate, and the informational material can be contained in a plastic sleeve or packet. In other embodiments, the separate elements of the kit are contained within a single, undivided container. For example, the composition or graft is contained in a container or culture plate that has attached thereto the informational material in the form of a label. The containers of the kits can be air tight, waterproof (e.g., impermeable to changes in moisture or evaporation), and/or light-tight.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Exemplary Plastination Protocol and Methods for Tissue Engineering

Step 1: Obtain a Decellularized Tissue.

A desired tissue, e.g., breast or another dermal tissue, is treated under conditions whereby it is substantially or completely decellularized and the resultant decellularized “tissue” retains the basic architecture of the tissue and comprises some or all the extracellular matrix proteins comprised in the tissue. Generally, these treatment methods will remove the skin, fat, and fibrous tissues from the tissue which is then treated to remove the remaining cells by a decellularization process by a means which preserves the basic architecture of the tissue and preferably retains extracellular matrix proteins comprised in the tissue. An exemplary procedure is the decellularization protocol contained in U.S. patent application Ser. No. 15/523,306, the entirety of which is incorporated herein by reference. This exemplary procedure includes a dehydration step using a dehydrating solvent, e.g., ethanol. Of course, when lyophilized or dried tissue is used to produce the plastic impregnated graft the dehydration step is not required. Also, as noted previously, the invention embraces the use of other decellularization procedures, i.e., other methods which substantially preserve the gross structure and/or architecture of the tissue or organ prior to decellularization and/or which result in decellularized tissues or organs which may be used to derive PPGs according to the invention.

Step 2: Optionally, fix the tissue. For example, the tissue can be fixed in an aldehyde, such as formaldehyde or glutaraldehyde.

Step 3: Submerge the tissue in a dehydrating solvent followed by a rehydrating solvent to ensure the water within the tissues is replaced. The tissue can be submerged in the solvent in a series of baths to ensure the water within the tissues is replaced using a rehydrating solvent. The water content in the baths can be measured before the tissue is submerged and at different timepoints while the tissue is submerged to determine when this step is complete. When all or substantially all of the water is removed from the bath, the process is complete.

As afore-mentioned exemplary dehydrating solvents include ethanol and other alcohols and rehydrating solvents include organic compounds such as acetone that become gaseous under vacuum. For example, rehydration can be effected by submerging the tissue in a cold acetone bath (for example approximately −15° C.-25° C.) for a sufficient period of time. The skilled artisan will recognize that time and temperature of the solvent baths will vary depending upon the polymer to be used, whether or not cells are used to repopulate the tissue, among other factors.

Optionally, solvent baths can be used to dissolve soluble body fat within the tissue. For example, acetone can be warmed to room temperature to dissolve soluble body fat. This step may be omitted

Step 4: Forced Vacuum Impregnation. The sample is submerged in a bath of a polymer or resin and subject to vacuum. The process is complete when no bubbles are observed.

The skilled artisan will recognize that a variety of polymers and/or resins can be used. For example, medical grade silicone can be used.

Different types of polymers can be utilized which encourage different types of cell growth. For example, alginate can be used as the polymer to stimulate neurite growth.

In embodiments, polymers can be mixed with other components, such as cells or antibiotics. For example, polymer mixed with cells can be used to seed new cells for tissue engineering application throughout biomaterials. At present, this is an unmet challenge for tissue engineering. As another example, polymers can be mixed with therapeutic and/or prophylactic agents such as pain relievers, anti-inflammatories, antibiotics, antifungals, or antimicrobials, allowing for sustained release of the therapeutic and or prophylactic agent.

In embodiments, the polymer can be biodegradable polymer (such as chitosan, collagen, alginate, cyanoacrylate polymers such as dermabond and like) or non-biodegradable polymers (such as silicon or ultra-high-molecular-weight polyethylene). Silicon, for example, can be used for soft tissues grafts, and UHMWPE can be used for bone grafts.

Step 5: Cross-Linking

The skilled artisan will recognize that a variety of cross-linkers and/or cross-linking methods can be used. For example, UV, chemical or gaseous cross-linking can be used, such as after the plastination protocol is complete, or slow cross-linking can be used, such as during the plastination protocol. For example, the cross-linker can be mixed with the polymer (for example, alginate, calcium carbonate, and GDL).

### Alternative Embodiments

If the tissue is too dense after the plastination process, such as too dense for cell seeding or too dense for a patient's own cells to migrate through the graft, embodiments can comprise utilizing higher concentration of water mixed with the polymer bath, less cross-linking density, and/or create holes or channels (such as mechanically, laser etched, chemically, light, and the like). Alternatively the addition of caustic agents such as sodium hydroxide and hydrogen peroxide can make the tissue less dense. Yet alternatively or additionally metal chelating agents and enzymes can be used to partially dissociate the tissue, allowing it to be more porous.

### Purposes and Uses:

I) Mechanical: Currently one of the major problems with decellularized matrices in the medical fields is reduced mechanical properties. By applying plastination to decellularized soft tissues, mechanical properties would be greatly modified (i.e., improved). For example, the polymers themselves could self-cross-link with the extracellular matrix, or cross-linking the extracellular matrix to the polymers or cross-linking the polymers to itself would allow for modification of mechanical properties. For example, more crosslinking can result in higher mechanical properties, such as young modulus. Polymers that act as weeping lubrications could also be used in decellularized cartilage or like materials such as joints or discs. Weeping lubricants refers to fluid that is squeezed out mechanically from cartilage or similar implants to maintain a layer of fluid on the cartilage or implant surface, so as to reduce friction and lubricate joints.

II) Cell Seeding: A hurdle within tissue engineering is delivering cells throughout the entire material or scaffold. Currently, some inject the cells into particular spots, create the material with cells before cross-linking for a physical encapsulation within the material (material limited) and push the cells through via fluid devices, seed on surface and allow for self-migration. Embodiments as described herein will utilize plastination for cell seeding, which will allow for cells to be seeded deeply and potentially evenly into the tissues. Such embodiments will be useful for materials that cannot be created premixed with cells, for physical encapsulation or when using whole organ decellularized scaffolds, such as lung, heart liver, pancreas.

In whole-organ biologically-derived scaffolds, for example, a common method of cellular seeding is to wash cells through the decellularized vessel networks or airways, and relying on self-migration of the cells to get into the walls and deeper parts of the whole organ scaffold. As described herein, by infusing and submerging the whole organ with polymers that have cells mixed therein, a substantially uniform distribution and thorough cellular seeding of the biologically derived scaffold will occur, even within the vessel walls and deeper of the whole organ scaffold. Vacuum pressure and duration, temperature process and solvents will be cell dependent.

III) Cell Migration/Differentiation: Different types of polymers or other compounds such as growth factors could be utilized to encourage different types of cell growth or migration of different cells into the tissue such as angiogenic factors, VEGF, cell-matrix adhesions, the Rho family of small GTPases, and other proteases which promote and regulate 3-D cell migration, among others. For example, alginate, can encourage neurospheres to differentiate into proportionately different subsets of cells than agarose, agar or gellan gum. Embodiments herein capitalize on these innate properties of different polymers to encourage different cells or subset thereof to grow and/or differentiate in some embodiments without the use of drugs or exogenous growth factors.

### Example 2

Pelvic organ prolapse (POP) is a common condition in women caused by loss of pelvic floor tissue support, causing organs to herniate into the vaginal lumen or anus. POP symptoms, like incontinence, frequent urinary tract infections, difficult bowel movements, bleeding, pressure, and pain, can have significant negative impacts on women's health, daily living, and quality of life, especially impacting body image, family relationships, and sexual health¹⁻³. Given the negative effects of untreated POP and the known risks associated with POP surgical treatment, POP represents an area of significant unmet medical need that is desperate for a safe and effective solution.

While ABGs are currently being developed for use in POP surgeries improved ABGs are needed. Through tissue engineering methods, grafts produced according to the invention will have enhanced mechanical properties and will serve as a drug delivery device for delivering various therapeutic agents, such as pain medication, antibiotics, anti-infectives, combinations of such. This graft will address POP's unmet medical need, as the inventive grafts should provide a safer, more effective product than synthetic mesh implants and ABGs currently on the market. In exemplary embodiments ABGs can be naturally derived from porcine or bovine dermal matrix that was made acellular and non-immunogenic through the process of decellularization. ABGs are ideal because they retain the native microenvironment of the extracellular matrix (ECM), which promotes host cell repopulation of the graft and, thus, complete integration of the graft with host tissue. Alternatively, ABGs obtained according to the invention can be derived using the patient's native tissues.

In POP, the vagina and supportive soft tissues are compromised both structurally and functionally, with these tissues showing disorganized and atrophied smooth muscle and altered collagen and elastin content^{30, 55-59}. As described herein, ABGs obtained using the inventive methods should provide safer options for POP repair relative to the current synthetic meshes and current ABGs.

ABGs are tissues, such as skin or tendon, that are recovered from a donor (human or animal) can be processed to remove cells and immunogens. ECM proteins (e.g. collagen, elastin), which largely comprise the architecture of most tissues, are retained during decellularization. This process essentially generates a cell- and DNA-free three-dimensional graft that is amenable to host cell repopulation and remodeling during the course of healing.

Non-autologous surgical material that can be used with the graft of the invention (1) provides structural support, (2) fosters the body's natural wound-healing process, (3) allows for ingrowth, and (4) fully integrates with host tissue in time. ABGs are superior to synthetic grafts in that they preserve, as oppose to recreate, the native matrix microenvironment, retaining the unique and complex matrix composition of the tissue^{20, 60}. Preserving these complex properties promotes efficient repopulation and functional re-establishment of cells and blood supply within the ABGs, leading to their increased success over synthetic grafts.

The ABGs according to the invention which are produced using decellularized donor tissue, e.g., dermal tissue from the recipient or another human donor or animal or dermal or other tissue made synthetically as afore-described will be impregnated with biodegradable polymers that can provide enhanced mechanical properties and local, sustained release of drugs. Without being bound by theory, polymer impregnation of an ABG will itself enhance the mechanical properties of the graft, allowing it to be more durable than a non-impregnated graft and thus prevent graft failure due to mechanical forces. Infection, commonly a result of opportunistic bacteria, could be offset through sustained, local antibiotic release from the polymer, while the addition of anti-inflammatory and analgesic agents could reduce local inflammation and decrease pain at the surgical site. These additive properties will increase graft acceptance and host-integration, lessening the likelihood of complications and easing patient recovery.

The innovation as described herein is the adaptation of tissue plastination techniques⁶³⁻⁶⁶ for mechanically strengthening ABGs and for sustained and localized delivery of drugs from these grafts. Polymer embedded grafts have been described previously; however, these are usually a combination of synthetic or natural polymers and fabricated collagen matrices⁶⁷; not naturally occurring ABGs. The inventors' method will force-impregnate polymers into the dense, native three-dimensional environment of an ABG, allowing for enhanced mechanical properties of the graft while retaining the morphological complexity of the endogenous ECM. Maintenance of the natural collagen fiber density and alignment within polymer-impregnated grafts will further provide support for robust recellularization and wound healing^{67, 68}.

### Rationale.

There are three main aspects to the polymer impregnation of ABGs as described herein: (1) decellularization of intact tissue, (2) plastination-based impregnation of natural polymers, and (3) drug release from polymers. In a first instance, aspects (1) and (2) as described will be integrated. In a second instance, the inventors will then incorporate (3) drug release.

(1) Decellularization: Decellularization techniques for whole organ regeneration of lung have been optimized in rat and non-human primate (NHP) models^{69, 70}, and have now successfully been employed on human skin. With modification of the decellularization process, feasibility of decellularizing dermal matrices has now been demonstrated⁷¹. The decellularization process meets previously defined criteria for generating non-immunogenic acellular tissue post decellularization^{21, 72}. This decellularization method is covered under US patent application no. US 2018/0015204 A1²⁶, which is hereby incorporated by reference in its entirety.

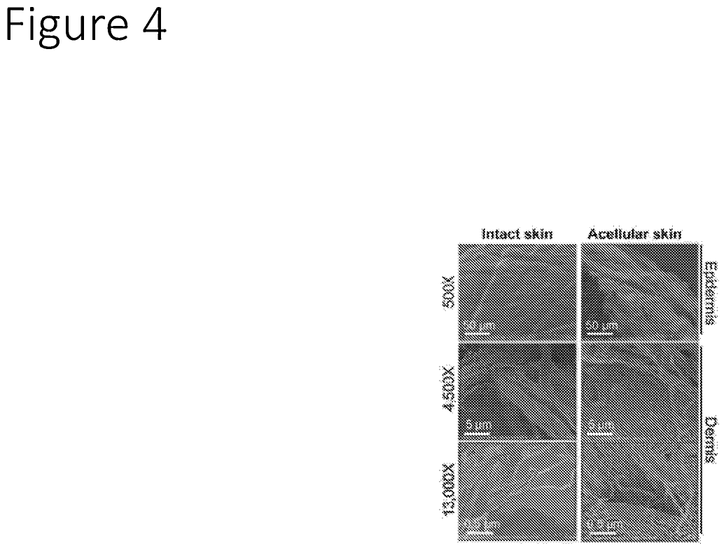

The decellularization process has been characterized on NHP dermis for the retention of ECM components collagen, glycosaminoglycans (GAGs), and elastin⁷¹. Briefly, collagens (types-I and -III) are major ECM components of skin, GAGs are hydrophilic polysaccharides that provide impact retention to the ECM, and elastin fibers are an essential component for skin elasticity. The inventors confirmed preservation of collagen and GAG content to levels similar to intact dermis, and detected a decrease in elastin levels⁷¹. A decrease in elastin is highly common in decellularized tissue^{73, 74}, however, the decreased elastin levels measured are sufficient for the maintenance of native-like mechanical properties^{23, 74}. The levels of retained ECM components from our decellularized NHP dermal matrix are similar to data from other epithelial tissue such as lung from rhesus macaque and rat, and skin from pig⁷³⁻⁷⁶. Furthermore, the decellularized dermis supported cell proliferation (>65%), cell migration, and did not significantly induce apoptosis (<1.5%)⁷¹. Additionally, the inventors evaluated ECM fiber structure of decellularized NHP dermis with scanning electron cryomicroscopy and found the integrity of collagen bundles and fibers were maintained in the dermal and epidermal layers, similar to intact dermis (FIG. 4). The decellularized dermis is devoid of immunogenic levels of genomic material and retains its overall structure on the micro- and macroscale. These data demonstrate that the decellularization process (without polymer encapsulation) is appropriately optimized for skin, creating an acellular matrix that retains structural elements.

(2) Plastination: Gunther von Hagens popularized plastination in the 1970s and the process has since been used to preserve surgical or autopsy tissue samples for teaching, histology, court evidence, and whole organism preservation⁶⁴. The process is well-understood⁶³⁻⁶⁶ with the most characterized method of polymer impregnation for human tissue being the S10 Technique^{65, 66}. In brief, this technique dehydrates fixed whole organs or organ slices through controlled incubations in organic solvents, including ethanol and acetone. Upon application of a vacuum, the acetone within a tissue undergoes a phase transition from liquid to gas, creating a negative pressure within the tissue that drives bath solutions, such as polymers, directly into the graft. This technique has been widely used with a range of polymers of various molecular sizes and charges, including silicones, polyesters, and resins⁷⁷⁻⁸¹. To date, plastination has not been used for the purpose of impregnating ABGs with polymers for use or in any therapeutic application.

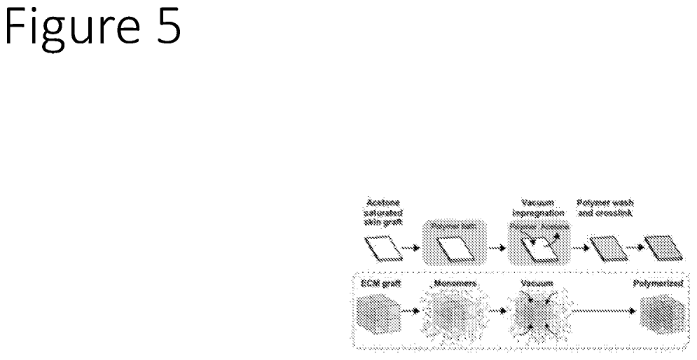

Changes to the standard S10 Plastination Technique will be made in order to impregnate ABGs with polymers for use as well as for therapeutic applications. The inventors will use the decellularization process described above to generate a native, non-crosslinked, ABG. After dehydration of the ABG through successive incubations in ethanol, the graft will be saturated with acetone. Similar to the original plastination process, the inventors will use controlled vacuum driven force impregnation²⁷ of the graft to replace the acetone in the graft with biomaterials and biocompatible biologically-derived polymers and proteins (FIG. 5).

Drug Release: Polymers, including biodegradable polymers, have long been used in drug delivery systems as carriers⁸². The inventors will utilize natural polymers that are amenable to drug encapsulation for localized drug delivery at sites of graft placement. The inventors will leverage knowledge and methods of dermal delivery and sustained-release of drugs that is well known in the art to develop a method to provide sustained drug release within regenerating tissue. This approach avoids systemic effects of current methods and provides a higher therapeutic dose at the relevant site of drug action. Articles have published relating to biodegradable polymers used to encapsulate and release growth factors in a controlled, sustained manner⁸³ (incorporated by reference in its entirety). This study used a novel alginate construct as a multi-functional tissue scaffold for central nervous system repair that delivered a brain-derived neurotrophic factor (Neurotrophin-3)⁸³. In the polymer-impregnated ABG program described herein, the inventors will use similar materials and methods to physically encapsulate antibiotics, analgesics, and/or anti-inflammatory agents in the polymers selected, to address issues with infection, pain, and host hyperinflammatory response.

### Analysis

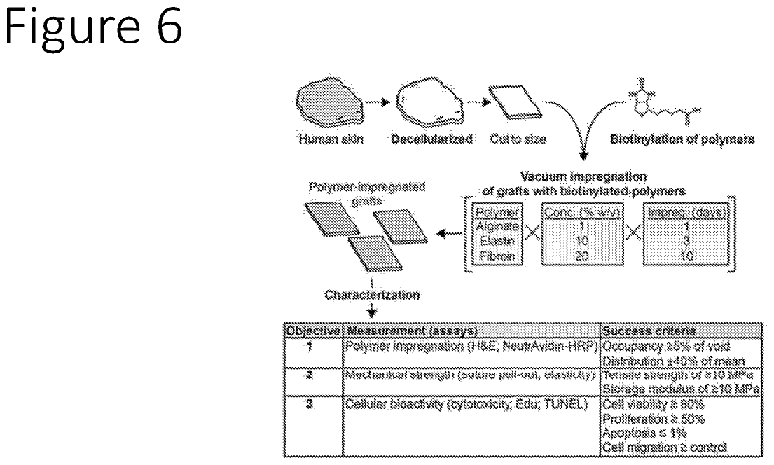

The inventors are developing a method to impregnate an ABG with biodegradable polymers that have well-characterized, robust mechanical properties and are amenable to the encapsulation of drugs for local and controlled delivery of compounds at engraftment sites. A polymer-impregnated graft will have enhanced mechanical properties, enabling it to be more durable than a non-impregnated graft and will decrease the likelihood of complications associated with mechanical failure. Controlled antibiotic release from these polymers at engraftment sites can have the potential to stem complications with infection and release of anti-inflammatory agents can immediately quell inflammation, allowing for increased likelihood of graft acceptance/integration with the host. Creation of a bioactive polymer-impregnated ABG requires complete decellularization of biologically-derived intact tissue, physical encapsulation of antibiotic and anti-inflammatory agents in a biodegradable polymer, and impregnation of the decellularized skin with the drug-loaded polymer. Additional efforts will be focused on developing and optimizing a novel polymer impregnation method based on tissue plastination techniques. The inventors will then analyze the polymer-impregnated ABGs' properties (FIG. 6). To that end, our objectives are:

Determine which Parameters Optimize Polymer Impregnation of ABGs.

The inventors will develop methods for impregnating acellular skin grafts with three biodegradable polymers: alginate, elastin, and silk fibroin. The three polymers will be tested at three concentrations each and three vacuum-impregnation incubation times will also be assessed. For each combination of parameters, the inventors will generate test grafts in triplicate. These grafts will be histologically analyzed for polymer penetration and distribution. The inventors will subsequently determine specific conditions that produce a polymer occupancy in the graft that is 5% of the graft void/interstitial space and have a polymer distribution such that normalized polymer occupancy is ±40% of the mean (for description, see Experimental Approach). Grafts that meet these success criteria will advance to Objectives 2 and 3 described herein.

The inventors will develop methods for impregnating acellular skin grafts with each of three biodegradable polymers: alginate, elastin, and silk fibroin. These well-characterized polymers were chosen for their biocompatibility, tunable mechanical properties, small and relatively simple monomeric form, ease of crosslinking to generate polymeric forms, biologically-derived nature, and proven use in biomedical applications. By impregnating ABGs with these polymers, the inventors can manipulate and enhance the mechanical properties of ABGs. In principle, the impregnated polymer can occupy a percentage of the void/interstitial space within the ECM and can crosslink onto itself and/or the ECM. Because these polymers readily crosslink through chemical (pH, Ca2+) or physical (temperature, sonication) mechanisms, crosslinking can occur rapidly and efficiently without regard for graft size. Non-enzyme-based crosslinking circumvents limitations with enzyme diffusion and sustained activity within the density of the graft. Additional reasons for selecting these three polymers are as follows:

(a) Alginate: Alginate (algin, alginic acid) is a natural polymer isolated from brown seaweed and is an FDA-approved polymer generally regarded as safe (GRAS). Alginate has been widely used in regenerative medicine and drug delivery, as it is histocompatible for human use and has minimal or negligible cytotoxicity⁸⁷⁻⁸⁰. As described in this section, alginate has been used as a tissue scaffold for drug release⁸³. Alginate can be induced to form highly cross-linked hydrogels with multivalent cations (e.g. Ca^{2±})⁸³.

(b) Elastin: The inventors have previously characterized a 69% reduction in elastin content in NHP acellular dermis from native dermis (n=3, p<0.01)⁷¹. These findings were similar to data from rhesus macaque lung, rat lung, and porcine dermis: elastin decrease during the decellularization process is a widely observed and characterized feature of detergent-based decellularization methods⁷³⁻⁷⁶. Recent studies looking at the addition of insoluble elastin in combination with collagen in a biomimetic cardiovascular tissue scaffold found that elastin markedly altered the mechanical and biological properties of the scaffold, reducing the specific tensile and compressive moduli without negatively affecting pore size or porosity⁹¹. Tropoelastin, the monomeric form of elastin, can be induced to crosslink with itself or collagen. The most commonly used elastin is α-elastin, which can be crosslinked by heat, repetitive sonication⁹¹, or pH change⁹². Alternatively, collagens (type I, III, IV, VII), fibronectin, and hyaluronic acid may be used.

(c) Silk Fibroin: Silk is primarily composed of two proteins, fibroin and sericin. Sericin may trigger an immune response, but fibroin does not⁹³. Silk has recently been explored as a drug delivery vehicle, drug stabilizer, and biological scaffold for tissue engineering⁹³ and is an FDA approved biomaterial. Silk is of interest to biomedical engineers due to its favorable biocompatibility properties and unique mechanical attributes, including a well-defined nano- and micro-scale structure hierarchy, biocompatibility, noninflammatory by-products, and sterilization method compatibility⁹³⁻⁹⁵. The FDA has approved a variety of silk products for use, including sutures and scaffolds (e.g. Seri® Surgical Scaffold). Silk can be induced to crosslink with itself or collagen by heat or repetitive sonication⁹³.

### Example 3

Acellular biologically-derived grafts (ABGs) are tissues recovered from donors and then decellularized to remove cells and immunogens. For 30+ years, ABGs have been used in surgeries for abdominal hernia repair, burn wound healing, diabetic ulcer/chronic wound repair, and other indications. ABGs have relatively high complication rates that are mainly attributed to infection, seroma formation, mechanical failure, and necrosis. These complications represent a significant unmet medical need for the 7.5 million Americans treated with ABGs annually.

Embodiments described herein address this unmet medical need, and include ABGs with tunable hydrogel-based drug delivery systems for sustained, local release of therapeutic agents from the ABG itself. Impregnating ABGs with polymers for therapeutic applications has not been performed before. Incorporating hydrogel-based drug delivery into ABGs allows for low-dose, single administration of drugs at targeted sites, thus decreasing drug toxicity and its metabolic breakdown.

Using a polymer impregnation technique described herein, drug-loaded hydrogel networks within the interstitium of human dermal ABGs that readily form hydrogel networks will enable the sustained and local release of therapeutic agents from the ABG. Validation experiments can include using vancomycin—a commonly used antibiotic for the treatment of Staphylococcus aureus infection—to demonstrate sustained release from ABGs impregnated with gelatin or silk fibroin polymer hydrogel networks. For example, validation studies can use dermal ABGs impregnated with natural, biodegradable polymers mixed with vancomycin. Embodiments herein utilizes force-driven displacement of a volatile solvent from a porous material, forcing polymers and drugs in the surrounding bath solution into the dense and diffusion-limited voids of the graft. Validation studies will (1) characterize ABG impregnation with vancomycin-polymer mixtures, (2) define drug-release profile and determine drug efficacy upon release from impregnated ABGs, and (3) characterize the cellular bioactivity of the impregnated ABGs.

These studies will validate the polymer-impregnated approach and validate the use of embodiments herein as a foundation for future drug-polymer-impregnated ABG research. Without wishing to be bound by theory, endowing ABGs with additive therapeutic properties by adding or incorporating additional actives such as antibiotics, analgesics, chemotherapeutics, anti-rejection agents, growth factors, antibodies, etc. wherein the active is comprised in the polymer matrix, e.g., a hydrogel or gelatin, will provide for the controlled release of desired actives and transform tissue engineering and regenerative approaches within the clinic. The selection of different hydrogels will provide for different release rates, e.g., which can be used to optimize the controlled “dosing” of actives comprised therein.

### Example 4

Acellular biologically-derived grafts (ABGs) are tissues recovered from donors and decellularized to remove cells, DNA, and immunogens. Extracellular matrix (ECM) proteins, which largely comprise the architecture of most tissues, are retained during this process, resulting in a cell- and DNA-free three-dimensional protein graft. For 30+ years, ABGs have been used in surgeries for abdominal hernia repair, breast reconstruction, burn wound healing, diabetic ulcer/chronic wound repair, and other indications^{1, 2}. Despite their advantages, ABGs have relatively high complication rates³⁻⁷ that are mainly attributed to infection, seroma formation, mechanical failure, and necrosis⁸⁻¹⁴. These complications are a significant unmet clinical need for the 7.5 million Americans treated with ABGs annually¹⁵.

To address these issues, embodiments herein comprise ABGs with tunable hydrogel-based drug delivery systems (DDS) (i.e. polymers) for sustained, local release of therapeutic agents (e.g. antibiotics, pain medications, and anti-inflammatories). Incorporating hydrogel-based drug delivery via polymers into ABGs allows for low-dose, site-specific administration of drugs, thus avoiding the toxicity risk associated with systemic administration. Impregnating ABGs with polymers for therapeutic applications has not been performed before.

Without wishing to be bound by theory, polymer-impregnated ABGs (polyABGs) loaded with drugs (drug+polyABGs) will directly address known complications of traditional ABGs and result in improved clinical outcomes. This is based upon ABGs' established natural regenerative capabilities¹⁶⁻¹⁸ for tissue reconstruction and recent therapeutic advances in hydrogel platforms for controlled drug delivery¹⁹. Using a polymer impregnation technique described herein, a human dermal drug+polyABG will be made with biocompatible, drugloadable polymers that readily form hydrogel networks to enable the sustained, local release of therapeutic agents from the graft.

Embodiments can comprise drug-loaded hydrogel networks within the interstitium of dermal ABGs. Polymer impregnation techniques described herein can utilize force-driven displacement of a volatile solvent from a porous material, forcing polymers and drugs in the surrounding bath solution into the dense and diffusion-limited voids of the ABG. Gelatin and silk fibroin, safe and natural biodegradable polymers with known drug delivery properties, will be used for the hydrogel networks. In an exemplary embodiment vancomycin can be used as the model drug which is a commonly used antibiotic for the treatment of post-operative Gram-positive bacterial infections.

### Conclusions

Surgical sites are highly susceptible to colonization by pathogenic bacteria such as Staphylococcus aureus (S. aureus); therefore, the use of local and systemic antibiotic administration is frequently prescribed to post-operative patients. However, these antibiotics are not fully effective due to limitations of the delivery methods (e.g. drug metabolism, inefficient delivery to relevant site, etc.). Abdominal hernia repairs have shown 25% infection rates that are nearly as high as the 27% hernia recurrence rate⁵⁵. Clinical studies using ABGs for breast reconstruction have shown a reduced infection rate compared to hernia repairs, but a rate that is still prevalent at 6.9%⁴¹. These complications result in graft failure and require repeat surgery, increasing the risk of further complications to the patient and increasing cost. Improved solutions are required to combat this significant unmet medical need.

The ideal graft is biocompatible and resistant to infection, thrombosis, necrosis, and mechanical failure. Embodiments herein comprise a dermal ABG impregnated with biodegradable, biocompatible, drug-loaded polymers (drug+polyABG) that readily form hydrogel networks to enable the sustained and local release of therapeutic agents from the drug+polyABG. Without wishing to be bound by theory, drug+polyABGs delivering antibiotics, analgesics, and/or anti-inflammatory agents will address issues with infection, pain, and host hyperinflammatory responses, respectively, thus increasing graft acceptance and host integration, lessening the likelihood of complications, and improving patient pain management, healing, and patient recovery time. Additionally, the impregnated polymers may enhance the drug+polyABGs' mechanical properties, namely elasticity and tensile strength.

Embodiments described herein will address the significant unmet medical need of ABG complications to improve clinical outcomes for the 7.5 million Americans treated with ABGs each year¹⁵. As described herein ABGs can be impregnated with drug-loaded polymers for therapeutic applications, a novel concept that has not been performed previously. Validation studies will use gelatin and silk fibroin polymers. Both polymers are generally regarded as safe (GRAS) by the FDA, non-immunogenic, biocompatible, and promote cell adhesion and proliferation⁶⁶; they possess relatively small and simple monomeric forms, are biologically-derived, and readily crosslink through chemical (pH, alcohol) and physical (temperature, sonication) mechanisms. Silk fibroin is also well known for its mechanical resilience^{61, 67, 68}.

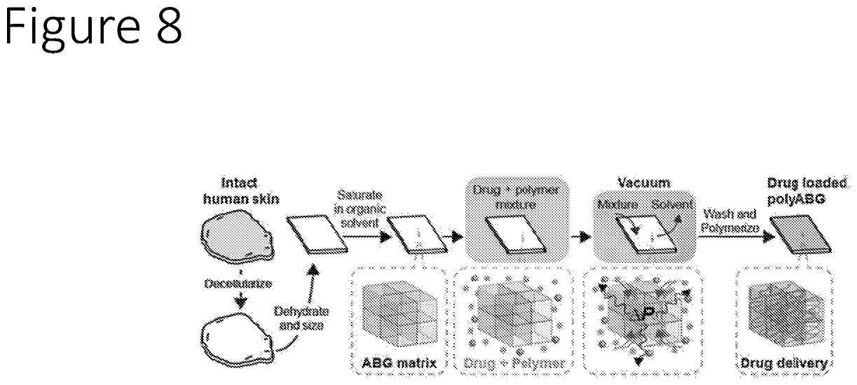

In exemplary embodiments an impregnation technique is used that is based upon force-driven displacement of volatile solvents, forcing polymers and drugs from the surrounding bath solution into the dense, diffusion-limited voids of the ABGs to create drug+polyABGs (FIG. 8). To the inventors' knowledge, impregnating ABGs with polymers for therapeutic applications has not been performed before.

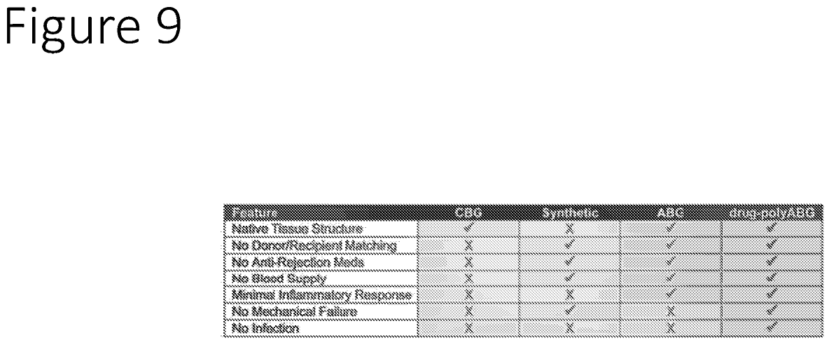

ABGs have not been previously used for hydrogel-based drug delivery systems, possibly due to the diffusion limited nature of the ECM. Our innovative approach overcomes this issue and drives polymers into the dense, native ECM of the ABG, allowing polymers to permeate the tissue interstitium (see data herein). Unlike polymer embedded grafts which attempt to recreate ECM, our polyABG approach utilizes actual ECM, harnessing its natural morphological complexity and cell amenable properties. FIG. 9 demonstrates the strengths of our drug+polyABG versus current options.

Framework:

The key steps involved in creating drug+polyABGs comprise 1) decellularization of donor tissue, 2) force-driven impregnation of hydrogel-forming natural polymers, and 3) hydrogel-based drug release from the drug+polyABGs.

1) Decellularization: Decellularization approaches are each optimized for specific tissue and greatly preserve the composition, structure and mechanical complexity of the ECM. Decellularization techniques for whole organ regeneration of lung in rat and non-human primate (NHP) models have previously been optimized^{74,75}, and have successfully employed these approaches on human skin to create human dermal ABGs²⁴. The decellularization process meets widely-accepted criteria for generating non-immunogenic acellular tissue post decellularization^{24, 25}. For example, the decellularization method can comprise that described in US 2018/0015204, which is incorporated herein by reference in its entirety. The decellularization process on NHP dermis for the retention of ECM components collagen, glycosaminoglycans (GAGs), laminin, fibronectin, and elastin has been characterized²⁴. The preservation of these molecules to levels similar to intact dermis has also been confirmed. The levels of retained ECM components from decellularized NHP dermis are similar to data from other epithelial tissue such as lung from rhesus macaque and rat, and skin from pig⁷⁷⁻⁸⁰. Furthermore, the decellularized NHP dermis supports cell proliferation (>65%), cell migration, and does not significantly induce apoptosis (<1.5%)²⁴. Lastly, experiments on NHPs with our decellularized human dermal ABGs have been completed. These ABGs support reepithelialization and neovascularization, showing near complete epithelial coverage at 6 weeks post-engraftment (91.4±8.6% coverage, n=6 ABGs). Neovascular formation, as measured by PECAM1+ vessel lumens, occurred within these ABGs as early as 1-week post engraftment, with more mature vessel formation, as well as increased presence of vessels, occurring over time. These data demonstrate the decellularization process is appropriately optimized for skin, creating a dermal ABG that supports regeneration.

2) Polymer Impregnation: The polymer impregnation concept herein can comprise those of industrial processes for fortifying porous materials such as wood, and plastination for preserving tissues and whole organisms⁶⁹⁻⁷². Both applications similarly involve replacement of water from samples with organic solvents possessing high vapor pressures. Upon vacuum application, the solvent within the sample becomes volatile and undergoes a phase transition from liquid to gas. This solvent transition and resultant evacuation from the sample creates a negative pressure within the sample that forces bath solutions, such as polymers and drugs, into the material. This technique has been used in industrial applications with a range of substances of various molecular sizes and charges⁸¹⁻⁸⁵.

The approach and application herein differs from prior polymer impregnation uses in several key areas. First, the application is for generation of a hydrogel-based drug delivery system for therapeutic purposes. This vastly differs from prior uses of polymer impregnation which have exclusively been used for material fortification and preservation. Second, embodiments herein are impregnating biomaterials with biocompatible, natural polymers. This greatly differs from prior uses of polymer impregnation in which polymers such as adhesives, resins, and alloys are used. Lastly, the sample material is an ABG, not a fixed cadaveric tissue or man-made material. Because the sample materials differ, the conditions for polymer impregnation do as well. Polymer impregnation conditions have been extensively optimized to be suitable for dermal ABGs. Impregnation conditions established for fixed, intact tissue do not result in impregnation of acellular tissue.

3) Hydrogel-based Drug Delivery: Natural biodegradable polymers have long been used in drug delivery systems (DDS) as carriers⁸⁶. They can form hydrogel networks that enable controlled release of hydrophobic and hydrophilic drugs in constant doses over long periods⁸⁶. Their biodegradable properties allow for gradual disappearance, eliminating removal of the drug carrier itself. Natural hydrogel DDS are fully tunable, allowing for control of their network pore size. This is important since the pores of the hydrogel will entrap the drug and, depending on the nature of the drug, pore size can be adjusted to physically entrap small versus large therapeutic agents. Gelatin and silk fibroin, for example, can be utilized in validation studies since they are well-established natural polymers that are amenable to drug loading and release⁵⁷⁻⁶⁵. Knowledge and methods of drug delivery and hydrogel-based drug delivery will be utilized to enable sustained drug release from an ABG over time. Biodegradable polymers used to encapsulate and release growth factors in a controlled, sustained manner has previously been shown⁸⁷. This study used a novel alginate construct as a multi-functional tissue scaffold for central nervous system repair that delivered a brain-derived neurotrophic factor (Neurotrophin-3)⁸⁷. In our drug+polyABG program, we will use similar methods to physically entrap vancomycin in gelatin and silk fibroin hydrogels.

Localized drug delivery is better suited for surgical site infections as it provides a higher therapeutic dose at the relevant site of drug action, avoids drug metabolism, and reduces off-target effects. A hydrogel-based ABG DDS will provide sustained, local release, allowing for low-dose, targeted, non-systemic administration for surgical site infections.

### Preliminary Data:

Through the polymer impregnation approach described herein, hydrogel networks within ABGs have been generated. Studies have identified impregnation conditions that lead to successful polymer impregnation within dermal ABGs, enabling slight to near complete impregnation of interstitial spaces. These studies were largely performed with natural polymers and, to a lesser extent, synthetic polymers. Preliminary data from both natural and synthetic polymers consistently demonstrate that polymer impregnation via force-driven displacement of a volatile solution from the ABG vastly outperforms diffusion alone. Gravity-based diffusion of polymeric solutions will penetrate hundreds of microns into the graft, exclusively occupying the perimeter of the graft whereas force-driven impregnation will lead to polymer occupancy throughout the entire graft, with penetration depths in the several of millimeters (FIG. 21). These data point to the natural diffusion-limited properties of the dermal ABG and how our polymer-impregnation approach overcomes this limitation. These validation studies demonstrate the ability to generate gelatin and silk fibroin hydrogels within ABGs.

### Example 5

In this Example a novel polymer impregnation method, based upon tissue plastination, is provided which may be used to generate an acellular biologic graft suited for use. To the best of the inventors' knowledge, plastination has not previously been used to impregnate acellular biologic grafts with polymers for therapeutic usage, particularly these procedures have not been used in human reconstructive therapy, e.g., breast reconstruction.

To improve clinical outcomes for POP procedures that use either acellular biologic grafts or synthetic meshes, the inventors will impregnate an acellular biologic graft with biodegradable polymers that can enhance the mechanical properties of the graft and provide local, sustained drug delivery. This method will incorporate the polymer into the natural three-dimensional environment of the acellular tissue, which retains the extracellular matrix, including naturally aligned collagen fibers, shown to support recellularization and wound healing. Creation of a bioactive polymer-impregnated acellular biologic graft will include complete or substantially complete decellularization of donor tissue, physical encapsulation of antibiotic and anti-inflammatory agents in a biodegradable polymer, and impregnation of the decellularized skin with the drug-loaded polymer. We have previously successfully decellularized a variety of tissues (lung, tumors, adipose, nipple-areolar complexes) including skin. In this Example, the inventors describe a new polymer impregnation method and characterize the mechanical properties and cellular bioactivity of the resultant grafts and teach encapsulating desired compounds within these biodegradable polymers, prior to graft impregnation. This will allow the graft to release drug in a sustained manner at engraftment/surgical sites.

In particular, the invention discloses methods for producing ABGs for treating POP, as afore-mentioned, an area of significant unmet medical need.

This invention should provide a new type of ABG for POP surgeries. This graft will have enhanced mechanical properties and serve as a vehicle for delivery of drugs such as pain medication, antibiotics, and anti-infectives. This graft will address POP's unmet medical need, as it should be a safer, more effective product than synthetic meshes and ABGs currently on the market. Particularly the inventors are combining a decellularization method²⁶ with a new bioactive polymer impregnation approach based on tissue plastination²⁷ to create a polymer-enhanced ABG. Tissue engineering approaches allow for the development of biologic substitutes that can replace or restore natural tissues. Specifically, we will use natural, biodegradable polymers engineered to release antibiotic, analgesic, or anti-inflammatory compounds, to prevent mechanical or immunological failure of an ABG. The polymers will enhance the mechanical properties of the ABG, namely elasticity and tensile strength, allowing it to be more durable than a non-impregnated ABG and, thus, prevent graft failure due to disruptive mechanical forces. These same polymers can encapsulate drugs for local release within the graft-surgical site. Release of antibiotic compounds can offset infection from opportunistic bacteria, improving healing and patient recovery time. Release of analgesic and anti-inflammatory compounds can decrease pain and local hyperinflammation, improving patient pain-management and increasing the likelihood of graft acceptance and host integration.

Here, the inventors discuss developing the plastination-inspired polymer impregnation method. We will then analyze the polymer impregnated ABGs' physical, mechanical, and bioactive properties.

ABGs are ideal because they retain the native microenvironment of the extracellular matrix (ECM), which promotes host cell repopulation of the graft and, thus, complete integration of the graft with host tissue.

The invention should provide a new type of ABG suitable for use in POP surgeries for women with severe POP. Through tissue engineering practices, our polymer-impregnated ABG will have enhanced mechanical properties and will serve as vehicle for delivering drugs such as pain medication, antibiotics, and anti-infectives, to help decrease potential complications and improve the safety and efficacy of POP surgery.

The inventors discuss herein the development of a new polymer-impregnated ABG for use in severe POP surgeries, which will be significantly safer and more effective than existing treatment options.

In POP, the vagina and supportive soft tissues are compromised both structurally and functionally, with these tissues showing disorganized and atrophied smooth muscle and altered collagen and elastin content^{30, 55-59}. Physicians are turning to ABGs as safer options for POP repair over the use of synthetic meshes. ABGs are tissues, such as skin or tendon, that are recovered from a donor (human or animal) and processed to remove cells and immunogens. ECM proteins (e.g. collagen, elastin), which largely comprise the architecture of most tissues, are retained during decellularization. This process essentially generates a cell- and DNA-free three-dimensional graft that is amenable to host cell repopulation and remodeling during the course of healing. However, improved ABGs are needed.

To improve clinical outcomes for POP procedures and to develop new capabilities of ABGs for wider applications in regenerative medicine, the inventors will impregnate ABGs with biodegradable polymers that can provide enhanced mechanical properties and local, sustained release of drugs. Without being bound by theory, polymer impregnation of an ABG will itself enhance the mechanical properties of the graft, enabling it to be more durable than a non-impregnated graft and thus prevent graft failure due to mechanical forces. Infection, commonly a result of opportunistic bacteria, could be offset through sustained, local antibiotic release from the polymer, while the addition of anti-inflammatory and analgesic agents could reduce local inflammation and decrease pain at the surgical site. These additive properties will increase graft acceptance and host-integration, lessening the likelihood of complications and easing patient recovery.

The inventors describe here in this example the adaptation of tissue plastination techniques⁶³⁻⁶⁶ for strengthening ABGs and sustained, localized delivery of drugs from ABGs. Polymer embedded grafts have been described; however, these are usually a combination of synthetic or natural polymers and fabricated collagen matrices⁶⁷; not naturally occurring ABGs. By contrast, in the current invention we force-impregnate polymers into the dense, native three-dimensional environment of an ABG, allowing for enhanced mechanical properties of the graft while retaining the morphological complexity of the endogenous ECM. Maintenance of the natural collagen fiber density and alignment within polymer-impregnated grafts will further provide support for robust recellularization and wound healing^{67, 68}.

There are three main aspects to the polymer impregnation of ABGs described herein: (1) decellularization of intact tissue, (2) plastination-based impregnation of natural polymers, and (3) drug release from polymers. Here, the inventors will integrate concepts (1) and (2). Also, the inventors will further incorporate (3) drug release.

Decellularization: Using previously optimized decellularization techniques for whole organ regeneration of lung in rat and non-human primate (NHP) models^{69, 70}, we have successfully employed these approaches on human skin. With modification of this decellularization process, the inventors have demonstrated feasibility of decellularizing dermal matrices⁷¹. We have shown that our decellularization process meets previously defined criteria for generating non-immunogenic acellular tissue post decellularization^{71, 72}. For example, see US 2018/0015204 A1 (“Surgical Grafts for Replacing the Nipple and Areola or Damaged Epidermis”)²⁶, which is incorporated by reference herein its entirety.

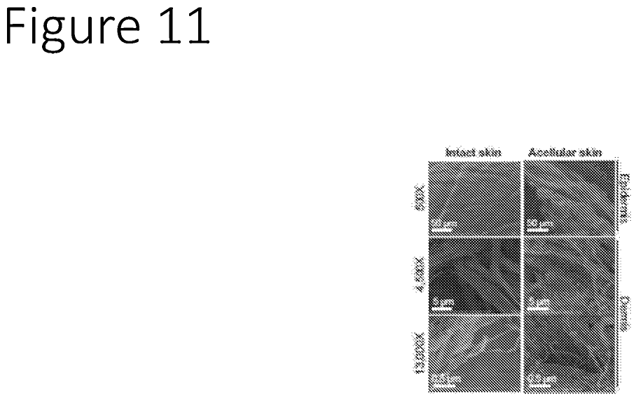

We have characterized our decellularization process on NHP dermis for the retention of ECM components collagen, glycosaminoglycans (GAGs), and elastin⁷¹. Briefly, collagens (types-I and -III) are major ECM components of skin, GAGs are hydrophilic polysaccharides that provide impact retention to the ECM, and elastin fibers are an essential component for skin elasticity. We confirmed preservation of collagen and GAG content to levels similar to intact dermis and detected a decrease in elastin levels⁷¹. A decrease in elastin is highly common in decellularized tissue^{73, 74}; however, the decreased elastin levels we measured are sufficient for the maintenance of native-like mechanical properties^{73, 74}. The levels of retained ECM components from our decellularized NHP dermal matrix are similar to data from other epithelial tissue such as lung from rhesus macaque and rat, and skin from pig⁷³⁻⁷⁶. Furthermore, our decellularized dermis supported cell proliferation (>65%), cell migration, and did not significantly induce apoptosis (<1.5%)⁷¹. Additionally, we evaluated ECM fiber structure of our decellularized NHP dermis with scanning electron cryomicroscopy and found the integrity of collagen bundles and fibers were maintained in the dermal and epidermal layers, similar to intact dermis (FIG. 11). Our decellularized dermis is devoid of immunogenic levels of genomic material and retains its overall structure on the micro- and macroscale. These data demonstrate that our decellularization process is appropriately optimized for skin, creating an acellular matrix that retains structural elements.

Plastination: Gunther von Hagens popularized plastination in the 1970s and the process has since been used to preserve surgical or autopsy tissue samples for teaching, histology, court evidence, and whole organism preservation⁶⁴. The process is well-defined63-66 with the most well-known and well-characterized method of polymer impregnation for human tissue being the S10 Technique^{65, 66}. In brief, this technique dehydrates fixed whole organs or organ slices through controlled incubations in organic solvents, including ethanol and acetone. Upon application of a vacuum, the acetone within a tissue undergoes a phase transition from liquid to gas, creating a negative pressure within the tissue that drives bath solutions, such as polymers, directly into the graft. This technique has been widely used with a range of polymers of various molecular sizes and charges, including silicones, polyesters, and resins⁷⁷⁻⁸¹. To date, plastination has not been used for the purpose of impregnating ABGs with polymers for use in any therapeutic application.

Improvements will be made to the standard S10 Plastination Technique. First, we will use our decellularization process described above to generate a native, non-crosslinked, ABG. After dehydration of the ABG through successive incubations in ethanol, the graft will be saturated with acetone. Similar to the original plastination process, we will use controlled vacuum driven force impregnation²⁷ of the graft to replace the acetone in the graft with biomaterials and biocompatible biologically-derived polymers and proteins (FIG. 5).

Drug Release: Polymers, including biodegradable polymers, have long been used in drug delivery systems as carriers⁸². We plan to utilize well established natural polymers that are amenable to drug encapsulation for localized drug delivery at sites of graft placement. We will leverage knowledge and methods of dermal delivery and sustained-release of drugs to develop a method to provide sustained drug release within regenerating tissue. This approach avoids systemic effects of current methods and provides a higher therapeutic dose at the relevant site of drug action. We have published on biodegradable polymers used to encapsulate and release growth factors in a controlled, sustained manner⁸³, which is incorporated by reference in its entirety. This study used an alginate construct, for example, as a multi-functional tissue scaffold for central nervous system repair that delivered a brain-derived neurotrophic factor (Neurotrophin-3)83. In our polymer-impregnated ABG program, we will use similar materials and methods to physically encapsulate antibiotics, analgesics, and/or anti-inflammatory agents in the polymers we select, to address issues with infection, pain, and host hyperinflammatory response.

Risks, along with some mitigation efforts, are summarized in Table 1 below.

TABLE 1

| Risk | Description | Mitigation |
|---|---|---|
| Polymer- | Size of protein | Use recombinant |
| impregnation | polymers hinder | polymer forms that |
| | penetration into tissue | retain polymerization |
| | Charge of polymers | to reduce monomer size |
| | exclude penetration | Solubilize polymers in |
| | into tissue | different pH solutions or |
| | | solvents to alter overall |
| | | charge state of polymers |
| Mechanical | Non-desirable | Optimize polymer |
| properties | mechanical | concentrations |
| | properties of | and include mild cross- |
| | tissue after | linking reagents to |
| | impregnation | covalently associate |
| | | polymers to matrix |
| Drug release | Temporally controlling | Optimize polymer |
| (Phase II) | release of drugs | concentrations to |
| | from polymeric | allow for steady |
| | encapsulation | drug release properties |

Overview: POP is an area of high unmet medical need, given the 33-50% lifetime risk of developing POP for women and the 12% lifetime incidence of POP surgery for women^{8, 28, 42-44}. None of the current surgical treatment options for severe POP are acceptable: native tissue replacements frequently fail within 2 years24, 25, synthetic meshes are unsafe^{11, 12, 35, 36}, and current ABGs have high complication rates³⁷⁻⁴¹. ABG product failures include poor mechanical integrity, immune rejection, and infection¹⁵⁻²². The inventors are developing a new type of ABG product that will address these problems, using our patented decellularization²⁶ and plastination-based polymer-impregnation²⁷ processes. To date, plastination has not been used for the purpose of impregnating ABGs with polymers for use in any therapeutic application.

Specifically, we are developing a method to impregnate an ABG with biodegradable polymers that have well-characterized, robust mechanical properties and are amenable to the encapsulation of drugs for local and controlled delivery of compounds at engraftment sites. A polymer-impregnated graft will have enhanced mechanical properties, enabling it to be more durable than a non-impregnated graft and will decrease the likelihood of complications associated with mechanical failure. Controlled antibiotic release from these polymers at engraftment sites can have the potential to stem complications with infection and release of anti-inflammatory agents can immediately quell inflammation, allowing for increased likelihood of graft acceptance/integration with the host. Creation of a bioactive polymer-impregnated ABG requires complete decellularization of biologically-derived intact tissue, physical encapsulation of antibiotic and anti-inflammatory agents in a biodegradable polymer, and impregnation of the decellularized skin with the drugloaded polymer.

(a) Alginate: Alginate (algin, alginic acid) is a natural polymer isolated from brown seaweed and is an FDA-approved polymer generally regarded as safe (GRAS)87. Alginate has been widely used in regenerative medicine and drug delivery, as it is histocompatible for human use and has minimal or negligible cytotoxicity⁸⁸⁻⁹¹. As mentioned herein section, the team has previously used alginate as a tissue scaffold for drug release83. Alginate can be induced to form highly cross-linked hydrogels with multivalent cations (e.g. Ca2+)⁸³.

(b) Elastin: As discussed above in the Decellularization section, we have previously characterized a 69% reduction in elastin content in NHP acellular dermis from native dermis (n=3, p<0.01)71. These findings were similar to data from rhesus macaque lung, rat lung, and porcine dermis: elastin decrease during the decellularization process is a widely observed and characterized feature of detergent-based decellularization methods⁷³⁻⁷⁶. Recent studies looking at the addition of insoluble elastin in combination with collagen in a biomimetic cardiovascular tissue scaffold found that elastin markedly altered the mechanical and biological properties of the scaffold, improving durability without negatively affecting pore size or porosity⁹². Tropoelastin, the monomeric form of elastin, can be induced to crosslink with itself or collagen. The most commonly used elastin is α-elastin, which can be crosslinked by heat, repetitive sonication⁹², or pH change⁹³.

(c) Silk Fibroin: Silk is primarily composed of two proteins, fibroin and sericin. Sericin may trigger an immune response, but fibroin does not⁹⁴. Silk has recently been explored as a drug delivery vehicle, drug stabilizer, and biological scaffold for tissue engineering⁹⁴ and is an FDA approved biomaterial. Silk is of interest to biomedical engineers due to its favorable biocompatibility properties and unique mechanical attributes, including a well-defined nano- and micro-scale structure hierarchy, biocompatibility, noninflammatory by-products, and sterilization method compatibility⁹⁴⁻⁹⁶. The FDA has approved a variety of silk products for use, including sutures and scaffolds (e.g. Seri® Surgical Scaffold). Silk can be induced to crosslink with itself or collagen by heat or repetitive sonication⁹⁴.

### Example 6

### Exemplary Tissue Impregnation of Drug and Release Properties

The inventors devised an experimental strategy to introduce polymeric materials and dyes into acellular dermal grafts in a non-destructive manner. The impetus for this technology was to re-enforce acellular grafts with enduring materials of known mechanical resilience as it is common that biologic grafts, once implanted, experience decreasing tensile strength because of host-mediated recellularization and subsequent remodeling.

The dermis is predominantly composed of bundled collagen that is configured in dense and interconnected, hydrophilic fibers. The voids between these fibers (known as interstitia) are narrow, tortuous, and diffusion-limited. We hypothesized that if we could introduce polymers into an acellular dermal graft, we could generate polymeric networks within the interstitia. In turn, these networks could theoretically increase the tensile strength (stress:strain ratio) of the graft, endowing it with the ability to resist biologically-induced mechanical breakdown.

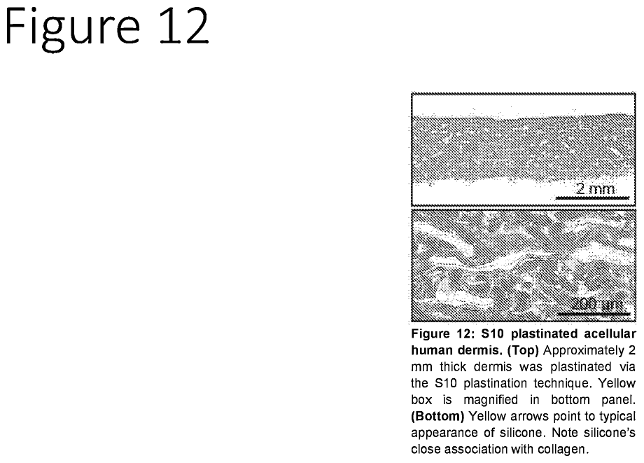

Our approach was primarily inspired by those used for plastination—the process of preserving tissue by replacing water within the sample with plastics (e.g. silicone). To this end, we performed plastination of acellular human dermal grafts. AlloMax® was used for most impregnation R&D efforts as it offered a cheap, fast and consistent starting acellular material to measure various impregnation conditions under. The beginning approach we used for plastination is generally referred to as the S10 technique which uses specialized silicone polymer and later curing of silicone with catalysts to ultimately enforce and preserve tissue samples. Successful impregnation of tissue with silicone via the S10 approach in our hands was validated by histological evaluation of tissue and with protease digestion. Staining with H&E showed silicone as an unstained, refractive, glass-like material present within the interstitia and having close association with neighboring collagen (FIG. 12).

FIG. 12 shows S10 plastinated acellular human dermis. In the top panel approximately 2 mm thick dermis was plastinated via the S10 plastination technique. Yellow box is magnified in bottom panel. In the bottom panel Yellow arrows point to typical appearance of silicone. Note silicone's close association with collagen.

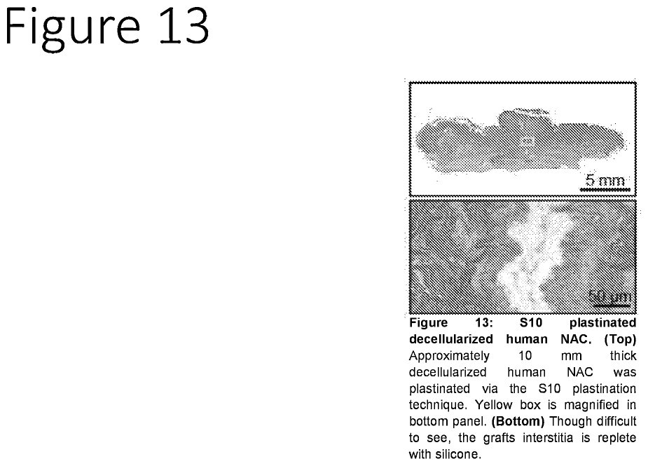

The results indicate that enzymatic digestion (50° C.) of plastinated tissue demonstrate its complete resistance to digestion with proteinase K over the span of weeks while non-plastinated tissue digested completely in <18 hours. The success of this approach was highly reproducible. Efforts were extended to plastination of human native and decellularized nipple areolar complex (NAC) grafts (FIG. 13). In these samples, we successfully impregnated highly dense and diffusion-limited interstitia with silicone that extended deep into the tissue. These samples largely resisted protease-based degradation.

FIG. 13 shows: S10 plastinated decellularized human NAC. In the top panel approximately 10 mm thick decellularized human NAC was plastinated via the S10 plastination technique. The yellow box is magnified in bottom panel. In the bottom panel, though difficult to see, the grafts interstitia is replete with silicone.

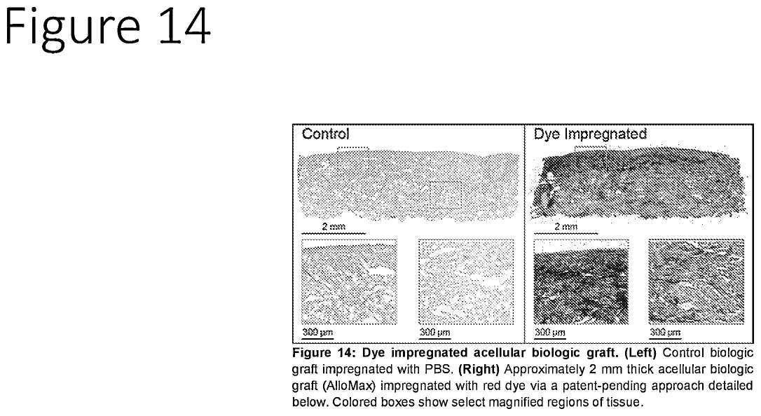

We experimented impregnating dyes for the purpose of having visual agents serve as a surrogate for impregnation efficacy since many of the polymers tested lacked histochemical detection. These limited efforts resulted in the presence of some, but not all, dyes producing a deep stain throughout AlloMax® samples. Some of the results from these experiments were derived from visual appearance and not histology. For example, impregnation of AlloMax® with Cibacron blue 3GA® showed a darker stained tissue as compared to AlloMax stained via diffusion of dye alone. Dextran blue (2MDa)®; however, did not show a similar visual appearance which may be due to the relatively large dextran particle. These samples, when halved, showed a stained cortex but the center of the tissue was unstained. By contrast, use of tattoo dyes produced favorable results (FIG. 14).

More particularly, FIG. 14 shows the dye impregnated acellular biologic graft. The left panel shows a control biologic graft impregnated with PBS. In the right panel approximately 2 mm thick acellular biologic graft (AlloMax®) impregnated with red dye. Colored boxes show select magnified regions of tissue.

Tattoo dye or tattoo ink may be seen by itself or as small aggregates in many cases, in clusters that are tightly associated with collagen bundles. Interestingly, incubation of AlloMax® samples with tattoo dye under atmospheric pressure (i.e. no vacuum), resulted in staining that was comparable to vacuum impregnated samples (not quantified). This suggests that diffusion alone with these thin samples (˜2 mm thick) may not necessitate vacuum impregnation. Taken together, these experiments revealed that tattoo dye staining of decellularized grafts is possible and that dye retention within these grafts is not strictly a cell-mediated process. This opens the possibility of pre-coloring acellular dermal grafts.

The pre-processing steps to dehydrate the tissue with various organic solvents and impregnate the tissue under various vacuum pressures and times led us to a reliable method that potentiates an acellular dermal graft to impregnation with natural hydrophilic polymers. Below is the general framework for this protocol which has been optimized on AlloMax dermal acellular biologic grafts. Special consideration should be made for tissue thickness and complexity when devising the times for each step (thicker tissue will require longer incubation times). The protocol was generally written for acellular biologic grafts (ABG); however, AlloMax® was predominantly used for all optimizations and testing.

### General Impregnation Protocol:

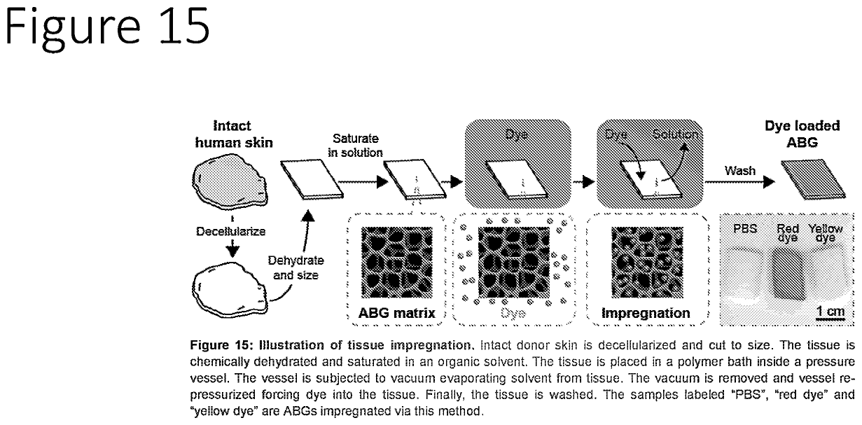

FIG. 15 is a schematic Illustration of an exemplary tissue impregnation according to the invention. As shown intact donor skin is decellularized and cut to size. The tissue is chemically dehydrated and saturated in an organic solvent. The tissue is placed in a polymer bath inside a pressure vessel. The vessel is subjected to vacuum evaporating solvent from tissue. The vacuum is removed and vessel re-pressurized forcing dye into the tissue. Finally, the tissue is washed. The samples labeled “PBS”, “red dye” and “yellow dye” are ABGs impregnated via this method.

### General Impregnation Protocol:

### Rehydration

1. Rehydrate lyophilized ABG with PBS under full vacuum (−25 inHg) for 15 minutes. Escaping air bubbles will cease once completely rehydrated.

### Chemical Dehydration

2. Incubate PBS-rehydrated ABG in a 25% ethanol bath for 1 hr at ambient temperature.

3. Incubate ABG in 50% ethanol bath for 1 hr at ambient temperature.

4. Incubate ABG in 75% ethanol bath for 1 hr at ambient temperature.

5. Incubate ABG in 100% ethanol bath for 1 hr at ambient temperature.

6. Incubate ABG in bath containing 50% ethanol and 50% acetone for 1 hr at ambient temperature.

7. Incubate ABG in bath containing 100% acetone indefinitely at −20° C. Cover with Parafilm or similar to decrease evaporative loss of acetone.

### Force-Impregnation

8. Pre-prepare polymer or dye solution for impregnation. Completely submerge dehydrated ABG in solution by introducing ABG to polymer or dye solution. Avoid carrying over excess acetone from ABG (i.e. it should not be dripping acetone).

9. Subject ABG to full vacuum for 1 hr. If the vessel is a capped-tube, it is recommended either a small hole is made in the cap of the tube or the tube is left open. For thicker samples, extend time under vacuum to >1 hr.

10. Degas ABG stepwise by moving ˜5 inHg lower in pressure every 10-15 minutes until atmospheric pressure is reached. This slow re-pressurization allows polymer or dye to be gradually drawn into the ABG. This step defines the act of impregnation.

11. Remove ABG and wash in PBS 1-2×. This step is not critical but recommended for cleaner histology results.

12. Pending on application, samples can be stored at 4° C. until use or fixed in 10% paraformaldehyde. If the polymer impregnated requires an active chemical polymerization step, the ABG can be subjected to conditions for this at this time.

Several variables were adjusted to identify the conditions above. These included changes to pressure, time under pressure, time during depressurization, tissue pre-processing conditions, and various polymers. The table below summarizes these efforts and the qualitative outcomes, as assessed by histological staining of impregnated tissue for polymer occupancy and distribution. The majority of optimization and testing was performed with silk fibroin, thus, the results stated largely reflect outcomes with this polymer. All vacuum pressures are negative values though not listed as such.

| | | Polymer | Polymer |
|---|---|---|---|
| | | occupancy | distribution |
| Condition | | (0-3) | (0-3) |
| Vaccum pressure | Low (5-10 inHg) | 2 | 1 |
| | High (20-25 inHg) | 3 | 3 |
| Time under vacuum | Fast (<10 min) | 1 | 1 |
| | Slow (60 min-overnight) | 3 | 2 |
| Time under degas | Fast (<10 min) | 1 | 1 |
| | Slow (60 min-overnight) | 3 | 3 |
| Pre-processing state | Lyophilized | 2 | 3 |
| | Water saturated | 0 | 0 |
| | Acetone saturated | 3 | 3 |
| | Acetaldehyde saturated | 2 | 3 |
| Polymer | Gelatin | 3 | 3 |
| | Silk fibroin | 3 | 3 |
| | Elastin | 1-3 | 1-3 |
| | Collagen | 1 | 1 |
| | Sylgard 184 | 2 | 2 |
| | Sylgard 170 | 1 | 1 |

### Vacuum Pressure

While maintaining time under vacuum, vacuum pressure was tested under low (5-10 inHg) and high (20-25 inHg) conditions. Low pressure is recommended for plastination with silicone and other hydrophobic and high viscosity polymers. This pressure ranges from 4-8 inHg. Low pressure impregnation worked for silk fibroin albeit not as well as high pressure (allowing vacuum to go to 25 inHg) which worked relatively better. It is important to note that by allowing complete evacuation of the ABG (air bubble release as acetone boils out), this step potentiates that tissue and allows for polymer to impregnate upon release of pressure and return to atmospheric conditions. The benefits of using lower pressure is to keep high vapor pressure agents within the impregnation at a lower temperature. Lower pressure also slow evacuation of solvent from tissue which decreases the amount of distortion to the tissue. The use of high pressure (25 inHg) is sufficient to impregnate dermal ABGs within a 1 hr period.

### Time Under Vacuum

Time while under vacuum is critical and, in addition to time under degas, should be increased for thicker and/or more complicated tissues. The prescribed 1 hr time under vacuum is longer than necessary for AlloMax® dermal ABGs. Roughly speaking, equal impregnation has been seen for impregnation times of 10 and 30 minutes. Because AlloMax® samples varied in thickness and collagen density, 1 hr was consistently used to account for this variability. It is worth noting here that the protocol above includes two separate vacuum-degas steps. The first step is optional; however, based on the manufacturer's instructions, rehydration of lyophilized AlloMax® in PBS for ˜30 minutes is not sufficient to completely rehydrate the sample. In tests, rehydrated AlloMax via this approach remains buoyant and, when placed under vacuum in the presence of PBS or water, escaping air bubbles are evident for ˜5 minutes when under 25 inHg. This suggests the ABG is not completely rehydrated which will likely have consequences with impregnation efficacy.

### Time Under Degas

This variable is similar to time under vacuum. For dense and complex tissue commonly used in plastination, longer vacuum times allow complete release of acetone while the degas step occurs relatively quicker than the time under vacuum. Step-wise degas over 1 hr is important in allowing polymer to make its way into the graft. The pressure differential between the inside of the graft and outside of the graft are uniform after the sample has been under vacuum for an extended period. Rapid degas causes the pressure outside of the graft to increase while the pressure inside the graft does so but not at the same rate. The goal of slow degas is to allow the viscous and still diffusion-limited polymer to gradually enter the tissue. Once return to atmospheric pressure has been reached, the polymer should have reached the depths of the graft as it migrated from the outside of the graft in.

### Pre-Processing State

This is an interesting area worthy of further investigation. Internal efforts showed that acetone saturated tissue is not the only form amenable to successful impregnation. Acetone and methyl chloride are the most commonly used for plastination. Although slightly different, xylene is used for impregnation for histology. Based on proof-of-concept experiments, it became clear that lyophilized material was capable of impregnation. This is important as many ABGs are packaged in a lyophilized state. Using a lyophilized ABG for impregnation would also be more amenable to impregnation with certain polymers and drugs that would otherwise be destroyed by harsh organic solvents. In most cases, impregnation of lyophilized tissue versus acetone saturated tissue was qualitatively similar. In the optimizations performed, the vast majority of work was performed with acetone saturated tissue. Acetone age (date opened) is not critical and did not show a difference from fresh acetone. Another interesting solvent that has a high vapor pressure and is natural is acetaldehyde. At this time, it is unclear if impregnation with acetaldehyde will enable impregnation.

### Polymer

Various polymers were experimented with. Initially, work with elastin (insoluble elastin and tropo-elastin (alpha-elastin)) was performed. Detection of elastin was performed with EVG and von Gieson staining. These efforts were inconsistent and, at times revealed increased elastin content and at other times, no difference. The outcomes from these efforts were not conclusive. Other polymers that have demonstrated success included agarose, gelatin, collagen (type-I), and silicone, to name a few. Silk fibroin was predominantly the polymer of choice. Impregnation of polymers is only as good as the ability to polymerize the polymer once within the tissue. Silk fibroin was readily polymerized with methanol. Agarose and gelatin were polymerized with cooling temperature. Elastin self-assembled. Collagen polymerized with a neutralizing solution of high alkalinity.

### Silk Fibroin Impregnation Protocol:

### Rehydration

1. Rehydrate lyophilized ABG with PBS under full vacuum (−25 inHg) for 15 minutes. Escaping air bubbles will cease once completely rehydrated.

### Chemical Dehydration

2. Incubate PBS-rehydrated ABG in a 25% ethanol bath for 1 hr at ambient temperature.

3. Incubate ABG in 50% ethanol bath for 1 hr at ambient temperature.

4. Incubate ABG in 75% ethanol bath for 1 hr at ambient temperature.

5. Incubate ABG in 100% ethanol bath for 1 hr at ambient temperature.

6. Incubate ABG in bath containing 50% ethanol and 50% acetone for 1 hr at ambient temperature.

7. Incubate ABG in bath containing 100% acetone indefinitely at −20° C. Cover with Parafilm or similar to decrease evaporative loss of acetone.

Force-Impregnation

8. Pre-prepare 5% silk fibroin (SF; Advanced BioMatrix) for impregnation. Completely submerge dehydrated ABG in SF by introducing ABG to SF solution. Avoid carrying over excess acetone from ABG (i.e. it should not be dripping acetone). For an 8 mm wide, 1 cm long, 2 mm thick dermal graft, placed in 1 mL of 5% SF solution.

9. Subject ABG to full vacuum for 1 hr. If the vessel is a capped-tube, it is recommended either a small hole is made in the cap of the tube or the tube is left open. For thicker samples, extend time under vacuum to >1 hr.

10. Degas ABG stepwise by moving ˜5 inHg lower in pressure every 10-15 minutes until atmospheric pressure is reached. This slow re-pressurization allows polymer or dye to be gradually drawn into the ABG. This step defines the act of impregnation.

11. Remove ABG and wash in PBS 1-2×. This step is not critical but recommended for cleaner histology results.

12. Expose the ABG to 1 mL of 80% methanol at room temperature. Allow the ABG to remained submerged in the methanol for 30 minutes before moving to 4° C.

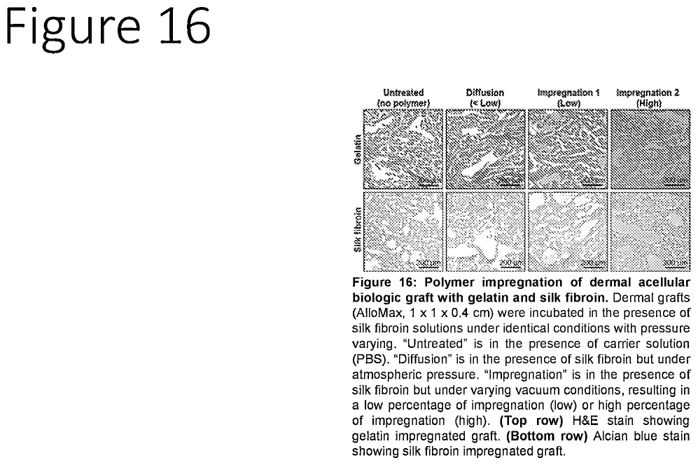

FIG. 16 shows results of the foregoing experiment wherein polymer impregnation of dermal acellular biologic graft with gelatin and silk fibroin. Dermal grafts (AlloMax®, 1×1×0.4 cm) were incubated in the presence of silk fibroin solutions under identical conditions with pressure varying. “Untreated” is in the presence of carrier solution (PBS). “Diffusion” is in the presence of silk fibroin but under atmospheric pressure. “Impregnation” is in the presence of silk fibroin but under varying vacuum conditions, resulting in a low percentage of impregnation (low) or high percentage of impregnation (high). (Top row) H&E stain showing gelatin impregnated graft. (Bottom row) Alcian blue stain showing silk fibroin impregnated graft.

### Drug Elution and Mechanical Testing:

The inventors sought to develop a drug+polyABG and, in turn, methods of generating them. By creating a polymer hydrogel within the ABG, we expect the mechanical properties of the ABG to be altered, and, by mixing the polymer with drugs, a hydrogel-based drug delivery system (DDS) can be created within the ABG to allow for sustained, local and/or intermittent release of therapeutic agents. Compared to conventional drug administration approaches which rely on high, repeated, systemic dosing, our drug+polyABG will provide reduced, sustained, local dosing. Release of antibiotics can offset infection from opportunistic bacteria, improving healing and patient recovery time. Release of analgesics and anti-inflammatory compounds can decrease pain and hyperinflammation, improving patient pain management and increasing the likelihood of graft acceptance and host integration. We expect the polymers to enhance the mechanical properties of the ABG, namely elasticity and tensile strength, enabling it to be more durable than a non-impregnated ABG and, thus, prevent graft failure due to disruptive mechanical forces.

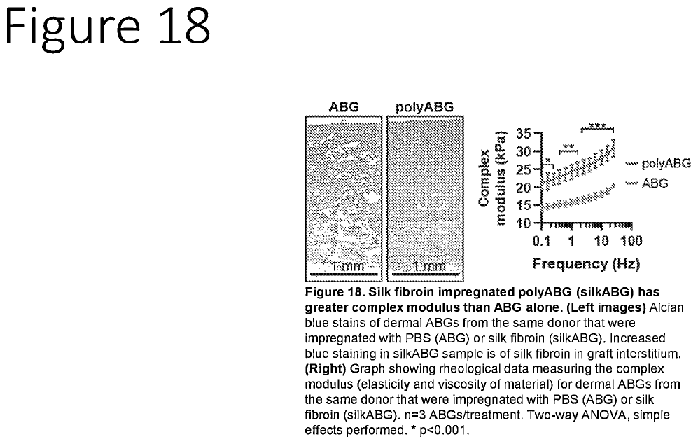

FIG. 17 contains data comparing silk fibroin impregnation of lyophilized versus acetone saturated dermal ABG. AlloMax was impregnated with either PBS or silk fibroin under identical conditions. AlloMax was either pre-processed and saturated in acetone or simply used in a lyophilized state.

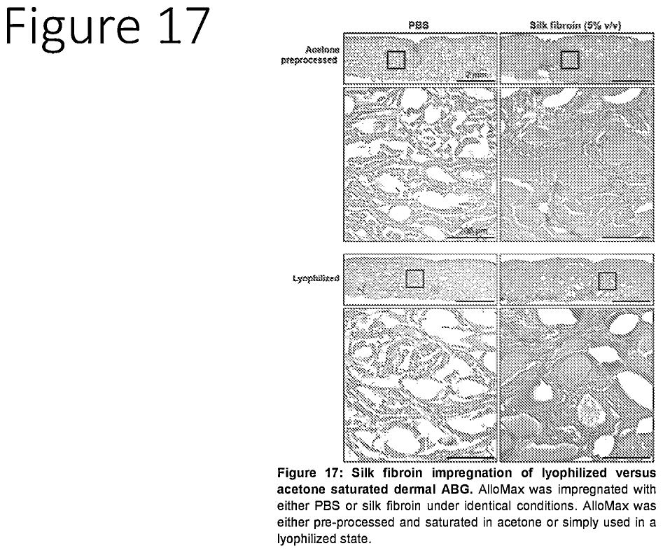

Rheological evaluation of silk fibroin impregnated polyABGs (silkABG) revealed an increase in complex modulus (i.e. the elasticity and viscosity of a material) over ABGs rehydrated with PBS (FIG. 18). In particular, FIG. 18 shows that silk fibroin impregnated polyABG (silkABG) has greater complex modulus than ABG alone. The left images contain Alcian blue stains of dermal ABGs from the same donor that were impregnated with PBS (ABG) or silk fibroin (silkABG). Increased blue staining in silkABG sample is of silk fibroin in graft interstitium. The right graph contains rheological data measuring the complex modulus (elasticity and viscosity of material) for dermal ABGs from the same donor that were impregnated with PBS (ABG) or silk fibroin (silkABG) (n=3 ABGs/treatment) and wherein two-way ANOVA, simple effects performed (*p<0.001).

This increase was well within the normal physiological stiffness for skin and suggests polyABGs can usefully enhance graft mechanical properties. Together, our preliminary data demonstrate our ability to generate silk fibroin hydrogels within ABGs. Measuring drug release kinetics is essential to determine if therapeutic potential of released drug can be achieved using our methods. Particularly drug release concentration, duration, and profile (i.e., continuous versus pulsatile) are measured. The kinetic profiles will show if drug release differs with polymer concentration. It is critical that drug release is sustained for the first two-weeks post engraftment since full reepithelialization of the ABG is not yet complete at that time and therefore the wound remains vulnerable to new infection.

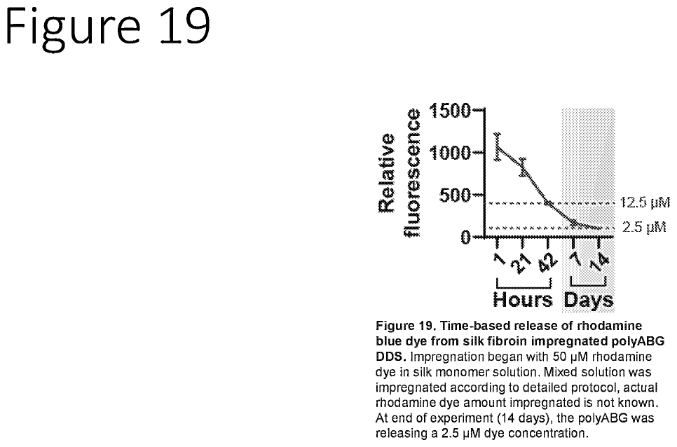

FIG. 19 contains experimental data demonstrating the time based release of rhodamine blue dye from silk fibroin impregnated polyABG DDS. As shown in these experiments impregnation began with 50 μM rhodamine dye in silk monomer solution. Mixed solution was impregnated according to a detailed protocol, however, the actual amount of rhodamine dye which is impregnated was not determined. At the end of the experiment (14 days), the polyABG was releasing a 2.5 μM dye concentration.

This data while preliminary demonstrates the ability of a silkABG DDS to maintain drug elution over the course of two weeks. Continued testing using the methods disclosed herein should permit the inventors to develop improved impregnation protocols which facilitate enhanced control of mechanical properties and drug release kinetics.

The contents of the references below are incorporated by reference in their entirety.

### REFERENCES

1. Lowenstein L, Gamble T, Sanses T V, van Raalte H, Carberry C, Jakus S, Kambiss S, McAchranS, Pham T, Aschkenazi S, Hoskey K, Kenton K, Fellow's Pelvic Research N. Sexual function is related to body image perception in women with pelvic organ prolapse. J Sex Med. 2009; 6(8):2286-2291.
2. Fritel X, Varnoux N, Zins M, Breart G, Ringa V. Symptomatic pelvic organ prolapse at midlife, quality of life, and risk factors. Obstet Gynecol. 2009; 113(3):609-616.
3. Chan S S, Cheung R Y, Yiu K W, Lee L L, Pang A W, Chung T K. Symptoms, quality of life, and factors affecting women's treatment decisions regarding pelvic organ prolapse. Int ETrogynecol J. 2012; 23(8): 1027-1033.
4. Barber M D, Maher C. Epidemiology and outcome assessment of pelvic organ prolapse. Int ETrogynecol 2013; 24(11): 1783-1790.
5. Aytan H, Ertunc D, Tok E C, Yasa O, Nazik H. Prevalence of pelvic organ prolapse and related factors in a general female population. Turk J Obstet Gynecol. 2014; 11 (3): 176-180.
6. Barber M D. Pelvic organ prolapse. BMJ. 2016; 354. doi: 10. H36/bmj.i3853.
7. “World Population 2018.” from https://countrymeters.info/en/World.
8. ETS Food and Drug Administration. ETrogynecologic Surgical Mesh: Update on the Safety and Effectiveness of Transvaginal Placement for Pelvic Organ Prolapse. 2011.
9. BBC News (2018). “Vaginal mesh implants: Australia apologises for ‘decades of pain’.” from https://www.bbc.com/news/world-australia-45806324.
10. Scott S, Branley A (2018). “Mesh implants: Government issues national apology over ‘agony and pain’ caused by device.” from https://www.abc.net.au/news/2018-10-10/mesh-implantsgoverment-issues-apology-to-women/10355546?section=health.
11. Kux L. Effective Date of Requirement for Premarket Approval for Surgical Mesh for Transvaginal Pelvic Organ Prolapse Repair. In: Food and Drug Administration, editor. Federal Register 2016. p. 364-370.
12. Schultz D G. FDA Public Health Notification: Serious Complications Associated with Transvaginal Placement of Surgical Mesh in Repair of Pelvic Organ Prolapse and Stress Urinary Incontinence. In: Center for Devices and Radiological Health, Food and Drug Administration, editors. 2008.
13. Campbell P, Jha S, Cutner A. Vaginal mesh in prolapse surgery. The Obstetrician & Gynaecologist. 2018; 20:49-56.
14. Margie Kahn. Personal Communication. 2018.
15. Weichman K E, Wilson S C, Weinstein A L, Hazen A, Levine J P, Choi M, Karp N S. The use of acellular dermal matrix in immediate two-stage tissue expander breast reconstruction. Plast Reconstr Surg. 2012; 129(5): 1049-1058.
16. Chun Y S, Verma K, Rosen H, Lipsitz S, Morris D, Kenney P, Eriksson E. Implant-based breast reconstruction using acellular dermal matrix and the risk of postoperative complications. Plast Reconstr Surg. 2010; 125(2):429-436.
17. Vig K, Chaudhari A, Tripathi S, Dixit S, Sahu R, Pillai S, Dennis V A, Singh S R. Advances in Skin Regeneration Using Tissue Engineering. Int J Mol Sci. 2017; 18(4).
18. MacNeil S. Progress and opportunities for tissue-engineered skin. Nature. 2007; 445(7130): 874-880.
19. Morris A H, Stamer D, Kyriakides T, editors. The host response to naturally-derived extracellular matrix biomaterials. Seminars in Immunology; 2017: Elsevier.
20. Shau D, Patton R, Patel S, Ward L, Guild G, 3rd. Synthetic mesh vs. allograft extensor mechanism reconstruction in total knee arthroplasty—A systematic review of the literature and meta-analysis. Knee. 2018; 25(I):2-7.
21. Purnell C A, Souza J M, Park E, Dumanian G A. Repair of recurrent hernia after biologic mesh failure in abdominal wall reconstruction. Am J Surg. 2014; 208(5):788-793.
22. Macadam S A, Lennox P A. Acellular dermal matrices: Use in reconstructive and aesthetic breast surgery. Can J Plast Surg. 2012; 20(2):75-89.
23. Future Market Insights. “Female Pelvic Implants Market: Companies to Focus on Reducing Cost of Implants to Gain Maximum Market Share: Global Industry Analysis 2012-2016 and Opportunity Assessment 2017-2027.” Market Report. 2018
24. Barber M D, Brubaker L, Burgio K L, Richter H E, Nygaard I, Weidner A C, Menefee S A, Lukacz E S, Norton P, Schaffer J, Nguyen J N, Borello-France D, Goode P S, Jakus-Waldman S, Spino C, Warren L K, Gantz M G, Meikle S F, Eunice Kennedy Shriver National Institute of Child Health, Human Development Pelvic Floor Disorders Network. Comparison of 2 transvaginal surgical approaches and perioperative behavioral therapy for apical vaginal prolapse: the OPTIMAL randomized trial. JAMA. 2014; 311(10): 1023-1034
25. Siff L N, Barber M D. Native Tissue Prolapse Repairs: Comparative Effectiveness Trials. Obstet Gynecol Clin North Am. 2016; 43(1):69-81.
26. Pashos N C, Bunnell B A, inventors; The Administrators of the Tulane Educational Fund, assignee. Surgical Grafts for Replacing the Nipple and Areola or Damaged Epidermis. U S patent US2018/0015204A1. Filed Oct. 31, 2015. Pub. Date Jan. 18, 2018.
27. Pashos N C, Heim W M, inventors; BioAesthetics Corporation, assignee. Polymer-Permeated Grafts and Methods of Making and Using the Same patent U.S. Provisional Patent Application 62/651,485. Filed Apr. 2, 2018.
28. Transparency Market Research. “Pelvic Organ Prolapse Devices Market—Global Industry Analysis, Size, Share, Growth, Trends and Forecast 2018-2026.” Market Report. 2018
29. Economics and Statistics Administration, US Census Bureau, US Department of Commerce. Age and Sex Composition: 2011.
30. Liang R, Knight K, Easley D, Palcsey S, Abramowitch S, Moalli P A. Towards rebuilding vaginal support utilizing an extracellular matrix bioscaffold. Acta Biomater. 2017; 57:324-333.
31. Pelley S (2018). “Gynecological Mesh: The Medical Device that has 100,000 Women Suing. “CBS 60 Minutes from https://www.cbsnews.com/news/boston-scientific-gynecological-mesh-themedical-device-that-has-100000-women-suing/.
32. Australia Co. Number of women in Australia who have had transvaginal mesh implants and related matters. 2018.
33. Perriello B (2018). “Johnson & Johnson's Ethicon loses $35 m pelvic mesh lawsuit.” from https://www.massdevice.com/johnson-johnsons-ethicon-loses-35m-pelvic-mesh-lawsuit/.
34. York M A (2018). “Ethicon, Inc. and J&J facing thousands of TVM and hernia mesh lawsuits: will they settle sooner or later?” from https://www.masstortnexus.com/mass-torts-news/ethicon-incand-jj-facing-thousands-of-tvm-and-hernia-mesh-lawsuits-will-they-settle-sooner-or-later/.
35. Therapeutic Goods Administration (2017). “TGA undertakes regulatory actions after review into urogynaecological surgical mesh implants.” From https://www.tga.gov.au/alert/tga-actions-afterreview-urogynaecological-surgical-mesh-implants.
36. Ismail S. Three countries ban the use of vaginal mesh in prolapse surgery. International Urogynecological Association 2018; 12(4).
37. Kim J Y, Davila A A, Persing S, Connor C M, Jovanovic B, Khan S A, Fine N, Rawlani V. A metaanalysis of human acellular dermis and submuscular tissue expander breast reconstruction. Plast Reconstr Surg. 2012; 129(1):28-41.
38. Ho G, Nguyen T J, Shahabi A, Hwang B H, Chan L S, Wong A K. A systematic review and metaanalysis of complications associated with acellular dermal matrix-assisted breast reconstruction. Ann Plast Surg. 2012; 68(4):346-356.
39. Loo Y L, Kamalathevan P, Ooi P S, Mosahebi A. Comparing the Outcome of Different Biologically Derived Acellular Dermal Matrices in Implant-based Immediate Breast Reconstruction: A Metaanalysis of the Literatures. Plast Reconstr Surg Glob Open. 2018; 6(3):e1701.
40. Trippoli S, Caccese E, Tulli G, Ipponi P, Marinai C, Messori A. Biological meshes for abdominal hernia: Lack of evidence-based recommendations for clinical use. Int J Surg. 2018; 52:278-284.
41. Slater N J, van der Kolk M, Hendriks T, van Goor H, Bleichrodt R P. Biologic grafts for ventral hernia repair: a systematic review. Am J Surg. 2013; 205(2):220-230.
42. Lawrence J M, Lukacz E S, Nager C W, Hsu J W, Luber K M. Prevalence and co-occurrence of pelvic floor disorders in community-dwelling women. Obstet Gynecol. 2008; 111(3):678-685.
43. Samuelsson E C, Victor F T, Tibblin G, Svardsudd K F. Signs of genital prolapse in a Swedish population of women 20 to 59 years of age and possible related factors. Am J Obstet Gynecol.1999; 180(2 Pt I):299-305.
44. Wu J M, Matthews C A, Conover M M, Pate V, Jonsson Funk M. Lifetime risk of stress urinary incontinence or pelvic organ prolapse surgery. Obstet Gynecol. 2014; 123(6): 1201-1206.
45. Market Reports World. “Global Pelvic Organ Prolapse Repair Device Market Report 2017” Market Report 2018
46. Brown L K, Fenner D E, Berger M B, Delancey J O, Morgan D M, Patel D A, Schimpf M O. Defining patients' knowledge and perceptions of vaginal mesh surgery. Female Pelvic Med Reconstr Surg. 2013; 19(5):282-287.
47. Weber A M, Walters M D, Piedmonte M R, Ballard L A. Anterior colporrhaphy: a randomized trial of three surgical techniques. Am J Obstet Gynecol. 2001; 185(6): 1299-1304; discussion 1304-1296.
48. Olsen A L, Smith V J, Bergstrom J O, Coiling J C, Clark A L. Epidemiology of surgically managed pelvic organ prolapse and urinary incontinence. Obstet Gynecol. 1997; 89(4): 501-506.
49. Kontogiannis S, Goulimi E, Giannitsas K. Reasons for and Against ETse of Non-absorbable, Synthetic Mesh During Pelvic Organ Prolapse Repair, According to the Prolapsed Compartment. Adv Ther. 2017; 33(12):2139-2149.
50. “Pelvic Organ Prolapse Repair Device Market Analysis, Market Size, Application Analysis, Regional Outlook, Competitive Strategies, and Forecasts, 2018-2025” Market Report. Market Expertz, 2018
51. Patrick J. Culligan C S, Christa Lewis, Troy D. Abell Cost-effectiveness analysis comparing robotic sacrocolpopexy to a vaginal mesh hysteropexy for treatment of uterovaginal prolapse. Open Journal of Obstetrics and Gynecology. 2013; 3:613-620.
52. “Wound Care Market and Market Share Analysis.” Market Report. Kalorama, 2017
53. “The Worldwide Market for Advanced Wound Care Products.” Market Report. Kalorama, 2018
54. Donate Life America (2018). “Tissue Donation: Everything You Need to Know.” from https://www.donateiife.net/types-of-donation/tissue-donation/.
55. Bildircin D, Kokcu A, Celik H, Sagir D, Kefeli M. Comparison of connective tissue components in the uterine ligaments between women with and without pelvic organ prolapse. Minerva Ginecol. 2014; 66(2):201-208.
56. Chen B, Yeh J. Alterations in connective tissue metabolism in stress incontinence and prolapse. J E Trol. 2011; 186(5): 17684772.
57. Meijerink A M, van Rijssel R H, van der Linden Pi. Tissue composition of the vaginal wall in women with pelvic organ prolapse. Gynecol Obstet invest. 2013; 75(I):21-27.
58. Boreham M K, Miller R T, Schaffer J I, Word R A. Smooth muscle myosin heavy chain and caldesmon expression in the anterior vaginal wall of women with and without pelvic organ prolapse. Am J Obstet Gynecol. 2001; 185(4):944-952.
59. Boreham M K, Wai C Y, Miller R T, Schaffer J I, Word R A. Morphometric analysis of smooth muscle in the anterior vaginal wall of women with pelvic organ prolapse. Am J Obstet Gynecol. 2002; 187(1):56-63.
60. Lintin L A, Kingsnorth A N. Mechanical failure of a lightweight polypropylene mesh. Hernia. 2014; 18(1): 13 1-133.
61. Jeffcoate W J, Vileikyte L, Boyko E J, Armstrong D G, Boulton A J M. Current Challenges and Opportunities in the Prevention and Management of Diabetic Foot Ulcers. Diabetes Care. 2018; 41(4):645-652.
62. Scarritt M E, Pashos N C, Bunnell B A. A review of cellularization strategies for tissue engineering of whole organs. Front Bioeng Biotechnol. 2015; 3:43.
63. von Hagens G, Tiedemann K, Kriz W. The current potential of plastination. Anat Embryol (Berl). 1987; 175(4):411-421.
64. Prasad G, Karkera B, Pandit S, Desai D, Tonse R G. Preservation of Tissue by Plastination: A Review. International Journal of Advanced Health Sciences. 2015; 1(11):27-31.
65. Riederer B M. Plastination and its importance in teaching anatomy. Critical points for long-term preservation of human tissue. J Anat. 2014; 224(3):309-315.
66. Latorre R M, Garcia-Sanz M P, Moreno M, Hernandez F, Gil F, Lopez O, Ayala M D, Ramirez G, Vazquez J M, Arencibia A, Henry R W. How useful is plastination in learning anatomy? J Vet Med Educ. 2007; 34(2): 172-176.
67. Castano O, Perez-Amodio S, Navarro-Requena C, Mateos-Timoneda M A, Engel E. Instructive microenvironments in skin wound healing: Biomaterials as signal releasing platforms. Advanced drug delivery reviews. 2018; 129:95-117. Epub 2018/04/09.
68. He C, Yang Z, Jin Y, Qi X, Chu J, Deng X. ADM Scaffolds Generate a Pro-regenerative Microenvironment During Full-Thickness Cutaneous Wound Healing Through M2 Macrophage Polarization via Lamtorl. Frontiers in physiology. 2018; 9:657. Epub 2018/06/20.
69. Bonvillain R W, Scarritt M E, Pashos N C, Mayeux J P, Meshberger C L, Betancourt A M, Sullivan D E, Bunnell B A. Nonhuman primate lung decellularization and recellularization using a specialized large-organ bioreactor. J Vis Exp. 2013(82):e50825.
70. Scarritt M E, Pashos N C, Motherwell J M, Eagle Z R, Burkett B J, Gregory A N, Mostany R, Weiss D J, Alvarez D F, Bunnell B A. Re-endothelialization of rat lung scaffolds through passive, gravity driven seeding of segment-specific pulmonary endothelial cells. J Tissue Eng Regen Med. 2018; 12(2):e786-e806.
71. Pashos N C, Scarritt M E, Eagle Z R, Gimble J M, Chaffin A E, Bunnell B A. Characterization of an Acellular Scaffold for a Tissue Engineering Approach to the Nipple-Areolar Complex Reconstruction. Cells Tissues Organs. 2017; 203(3): 183-193.
72. Crapo P M, Gilbert T W, Badylak S F. An overview of tissue and whole organ decellularization processes. Biomaterials. 2011; 32(12):3233-3243.
73. Bonvillain R W, Danchuk S, Sullivan D E, Betancourt A M, Semon J A, Eagle M E, Mayeux J P, Gregory A N, Wang G, Townley I K, Borg Z D, Weiss D J, Bunnell B A. A nonhuman primate model of lung regeneration: detergent-mediated decellularization and initial in vitro recellularization with mesenchymal stem cells. Tissue Eng Part A. 2012; 18(23-24):2437-2452.
74. Scarritt M E, Bonvillain R W, Burkett B J, Wang G, Glotser E Y, Zhang Q, Sammarco M C, Betancourt A M, Sullivan D E, Bunnell B A. Hypertensive rat lungs retain hallmarks of vascular disease upon decellularization but support the growth of mesenchymal stem cells, Tissue Eng Part A. 2014; 20(9-10): 1426-1443.
75. Hoganson D M, Owens G E, O'Doherty E M, Bowley C M, Goldman S M, Harilal D O, Neville C M, Kronengold R T, Vacanti J P. Preserved extracellular matrix components and retained biological activity in decellularized porcine mesothelium. Biomaterials. 2010; 3 1(27):6934-6940.
76. Price A P, England K A, Matson A M, Blazar B R, Panoskaltsis-Mortari A. Development of a decellularized lung bioreactor system for bioengineering the lung: the matrix reloaded. Tissue Eng Part A. 2010; 16(8):2581-2591. doi: 10. 1089/ten.TEA.2009.0659.
77. Chisholm F, Varsou O. Resin-embedded anatomical cross-sections as a teaching adjunct for medical curricula: is this technique an alternative to potting and plastination?J Anat. 2018; 233(498-105,
78. Ottone N E, Baptista C A C, Latorre R, Bianchi H F, Del Sol M, Fuentes R. E12 sheet plastination Techniques and applications. Clin Anat. 2018; 31 (5):742-756.
79. Rabiei A A, Esfandiary E, Hajian M, Shamosi A, Mardani M, Rashidi B, Setayeshmehr M. Plastination of decalcified bone by a new resin technique. Adv Biomed Res. 2014; 3: 18.
80. Soal S, Pollard M, Burland G, Lissaman R, Wafer M, Stringer M D. Rapid ultrathin slice plastination of embalmed specimens with minimal tissue loss. Clin Anat. 2010; 23(5): 539-544.
81. Miklosova M, Miklos V. Plastination with silicone method S10—monitoring and analysis causes of failure. Biomed Pap Med Fac ETniv Palacky Olomouc Czech Repub. 2004; 148(2):237-238.
82. Liechty W B, Kryscio D R, Slaughter B V, Peppas N A. Polymers for drug delivery systems. Annu Rev Chem Blomol Eng. 2010; 1: 149-173.
83. Shanbhag M S, Lathia J D, Mughal M R, Francis N L, Pashos N, Mattson M P, Wheatley M A. Neural progenitor cells grown on hydrogel surfaces respond to the product of the transgene of encapsulated genetically engineered fibroblasts. Biomacromolecules. 2010; 11(11):2936-2943.
84. Filipe E C, Santos M, Hung J, Lee B S L, Yang N, Chan A H P, Ng M K C, Rnjak-Kovacina J, Wise S G. Rapid Endothelialization of Off-the-Shelf Small Diameter Silk Vascular Grafts. JACC Basic Transl Sci. 2018; 3(1):38-53.
85. Pierce L M, Grunlan M A, Hou Y, Baumann S S, Kuehl Ti, Muir T W. Biomechanical properties of synthetic and biologic graft materials following long-term implantation in the rabbit abdomen and vagina. Am J Obstet Gynecol. 2009; 200(5):549 e541-548.
86. Musculoskeletal Transplant Foundation. Derma Matrix Acellular Dermis. Comparative testing, 2012.
87. ETS Food and Drug Administration. CFR—Code of Federal Regulations Title 21. 2018.
88. Sun J, Tan H. Alginate-Based Biomaterials for Regenerative Medicine Applications. Materials (Basel). 2013; 6(4): 1285-1309.
89. McCanless J D, Jennings L K, Bumgardner J D, Cole J A, Haggard W O. Hematoma-inspired alginate/platelet releasate/CaPO₄ composite: initiation of the inflammatory-mediated response associated with fracture repair in vitro and ex vivo injection delivery. J Mater Sci Mater Med. 2012; 23 (8): 1971-1981.
90. de Vos P, Spasojevic M, de Haan B J, Faas M M. The association between in vivo physicochemical changes and inflammatory responses against alginate based microcapsules. Biomaterials. 2012; 33(22): 5552-5559.
91. Vanacker J, Luyckx V, Dolmans M M, Des Rieux A, Jaeger J, Van Langendonckt A, Donnez J, Amorim C A. Transplantation of an alginate-matrigel matrix containing isolated ovarian cells: first step in developing a biodegradable scaffold to transplant isolated preantral follicles and ovarian cells. Biomaterials. 2012; 33(26):6079-6085.
92. Ryan A J, O'Brien F J. Insoluble elastin reduces collagen scaffold stiffness, improves viscoelastic properties, and induces a contractile phenotype in smooth muscle cells. Biomaterials. 2015; 73:296-307.
93. Mithieux S M, Tu Y, Korkmaz E, Braet F, Weiss A S. In situ polymerization of tropoelastin in the absence of chemical cross-linking. Biomaterials. 2009; 30(4):431-435.
94. Yucel T, Lovett M L, Kaplan D L. Silk-based biomaterials for sustained drug delivery. J Control Release. 2014; 190:381-397.
95. Horan R L, Toponarski I, Boepple H E, Weitzel P P, Richmond J C, Altman G H. Design and characterization of a scaffold for anterior cruciate ligament engineering. J Knee Surg. 2009; 22(1):82-92.
96. Wang Y, Rudym D D, Walsh A, Abrahamsen L, Kim H J, Kim H S, Kirker-Head C, Kaplan D L. In vivo degradation of three-dimensional silk fibroin scaffolds. Biomaterials. 2008; 29(24-25):3415-3428.
97. Polyak B, Geresh S, Marks R S. Synthesis and characterization of a biotin-alginate conjugate and its application in a biosensor construction. Biomacromolecules. 2004; 5(2):389-396.
98. Wang X, Kaplan D L. Functionalization of silk fibroin with NeutrAvidin and biotin. Macromol Biosci. 2011; 11(1): 100-110.
99. Manschot J F, Brakkee A J. The measurement and modelling of the mechanical properties of human skin in vivo—1. The measurement. J Biomech. 1986; 19(7): 511-515.
100. Ni Annaidh A, Bruyere K, Destrade M, Gilchrist M D, Ottenio M. Characterization of the anisotropic mechanical properties of excised human skin. J Mech Behav Biomed Mater. 2012; 5(1): 139-148.
101. American Society for Testing and Materials. ASTM D638-14, Standard Test Method for Tensile Properties of Plastics. West Conshohocken, Pa.: ASTM International; 2014.
102. American Society for Testing and Materials. ASTM D6797-15, Standard Test Method for Bursting Strength of Fabrics Constant-Rate-of-Extension (CRE) Bali Burst Test. West Conshohocken, Pa.: ASTM International; 2015.
103, Freytes D O, Rundell A E, Vande Geest J, Vorp D A, Webster Ti, Badylak S F. Analytically derived material properties of multilaminated extracellular matrix devices using the ball-burst test. Biomaterials. 2005; 26(27):5518-5531.
104. American Society for Testing and Materials. ASTM F543-17, Standard Specification and Test Methods for Metallic Medical Bone Screws. West Conshohocken, Pa.: ASTM International; 2014.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A polymer-permeated graft suitable for ex vivo or in vivo use in a subject, preferably human, comprising a tissue or organ substantially free of cells, wherein the decellularized tissue optionally is substantially free of water and is permeated with at least one polymer, preferably biocompatible, and further wherein the at least one polymer is substantially uniformly distributed in at least a substantial portion of said tissue or organ.

2. The graft of claim 1 which substantially retains the gross structure and/or architecture of the tissue or organ prior to decellularization.

3. The graft of claim 1 or 2 which further substantially retains the microarchitecture of the tissue or organ prior to decellularization.

4. The graft of any of the foregoing claims, wherein the graft comprises less than about 50% of polymer by weight.

5. The graft of any of the foregoing claims, wherein the graft comprises about 0.1% to about 30% of polymer by weight.

6. The graft of any of the foregoing claims, wherein the tissue or organ comprises dermal tissue and/or epidermal tissue.

7. The graft of any of the foregoing claims, wherein the tissue comprises an organ, a muscle, a ligament, a bone, a nipple, areola, a nipple attached to an areola, a lip, skin, a tendon, an aorta, a blood vessel and/or an amniotic membrane.

8. The graft of any of the foregoing claims, wherein the tissue substantially retains at least one matrix molecule.

9. The graft of any of the foregoing claims, wherein the matrix molecule comprises a component of the extracellular matrix.

10. The graft of any of the foregoing claims, wherein the matrix molecule comprises laminin, elastin, fibronectin, collagen, or a combination thereof.

11. The graft of claim 10, wherein the collagen comprises a Type I collagen, a Type III collagen, a Type IV collagen, a Type VI collagen, or a combination thereof.

12. The graft of any of the foregoing claims, wherein the tissue is substantially free of skin, fat and/or fibrous tissue.

13. The graft of any of the foregoing claims, which comprises a dye, pigment, ink or other colorant and/or the polymer comprises a colored polymer or comprises a polymer which changes in color under specific conditions and/or in the presence of specific moieties.

14. The graft of claim 13, wherein the colored polymer or colorant comprises melanin, a dye, an ink, e.g., tattoo ink, or a combination thereof.

15. The graft of any of the foregoing claims, wherein the polymer comprises a natural polymer and/or a synthetic polymer.

16. The graft of any of the foregoing claims, which comprises alginate and/or collagen.

17. The graft of any of the foregoing claims, which comprises a cyanoacrylate polymer.

18. The graft of any of the foregoing claims, wherein at least one active is comprised within at least one polymer which is comprised in the graft.

19. The graft of claim 18, wherein said active is selected from an analgesic, drug, anti-inflammatory, growth factor, hormone, antibiotic, chemotherapeutic, cytokine, anti-rejection agent, or a combination of any of the foregoing.

20. The graft of claim 18 or 19, wherein said active is comprised in a hydrogel polymer or other polymer which provides for controlled or sustained release of the active during in vivo use.

21. The graft of claim 20, which comprises different polymers respectively comprising different actives which are released at different rates.

22. The graft of any of the foregoing claims, wherein the graft comprises at least one viable cell and/or metabolically active cells, a polymer comprising at least one antibiotic, a biodegradable polymer, a non-biodegradable polymer, a polymer capable of cross-linking, chemotactic agent, or a combination thereof.

23. The graft of any of the foregoing claims, wherein at least one polymer in the graft comprises a biodegradable polymer.

24. The graft of claim 22 wherein said biodegradable polymer comprises chitosan, collagen, alginate, cyanoacrylate, and/or Dermabond™.

25. The graft of any of the foregoing claims, wherein the polymer is non-biodegradable and optionally comprises silicon and/or UHMWPE.

26. The graft of any of the foregoing claims, wherein the graft further comprises viable cell and/or metabolically active cells, optionally genetically engineered, wherein the cells have been introduced into the graft under conditions conducive to repopulate the tissue with the cells or progeny thereof.

27. The graft of claim 26, wherein the cells comprise exogenous cells, autologous cells, allogenic cells.

28. The graft of claim 26 or 27, wherein the viable cell and/or metabolically active cells comprise stromal cells, fibroblasts, endothelial cells, progenitor cells, stem cells, organ-specific cells, tissue-specific cells, keratinocytes, melanocytes, nerve cells, osteoclasts, or a combination thereof.

29. A method of making a polymer-permeated graft potentially for in vivo use comprising:
obtaining a decellularized tissue;
optionally dehydrating the tissue by submerging in at least one dehydrating solvent;
optionally, fixing the decellularized tissue by submerging the decellularized tissue in a fixative for a period of time sufficient to fix the tissue;
replacing substantially all of the water within the tissue with a solvent by submerging the decellularized tissue in a rehydrating solvent for a period of time and at a temperature sufficient to replace all or substantially all of the water within the tissue;
optionally, removing all or substantially all of the fat within the tissue by submerging the decellularized tissue in a solvent for a period of time and at a temperature sufficient to remove substantially all lipids;
permeating the tissue with a polymer which optionally is crosslinked, by submerging the tissue in the polymer and subjecting the submerged tissue to vacuum for a period of time sufficient to permeate the tissue with the polymer;
optionally, cross-linking the polymer permeated within the tissue;
wherein the polymer-permeated tissue comprises the polymer substantially uniformly distributed in at least a portion of said tissue;
thereby providing a polymer-permeated graft suitable for in vivo use.

30. The method of claim 29, wherein a chemical cross-linker has been admixed with the polymer prior to permeating the tissue.

31. The method of claim 29 or 30, further comprising decellularizing a tissue or organ comprising epidermal and/or dermal cells, while substantially retaining at least one matrix molecule.

32. The method of claim 29, 30 or 31, wherein the matrix molecule comprises laminin, fibronectin, elastin, collagen or a combination thereof.

33. The method of claim 32, wherein the collagen comprises a Type I collagen, a Type III collagen, a Type IV collagen, a Type VI collagen, or a combination thereof.

34. The method of any of claims 29-33, further comprising repopulating the tissue with viable cells and/or metabolically active cells under conditions conducive to repopulate the tissue with the cells or progeny thereof.

35. The method of claim 34, wherein the repopulating occurs at the same or about the same time as the permeating step and/or after said step.

36. The method of any of claims 29-35, wherein cell repopulating in the graft is effected after the permeating step.

37. The method of any of claims 34-36, wherein the cells comprise exogenous cells, autologous cells, allogenic cells.

38. The method of claim 37, wherein the cells comprise keratinocytes, melanocytes, a nerve cell, or a combination thereof.

39. The method of any of claims 29-38, wherein the fixative comprises glutaraldehyde, genipin.

40. The method of any of claims 29-39, wherein the rehydrating solvent comprises acetone, xylene or another solvent having a high vapor pressure and the dehydrating solvent comprises an alcohol, e.g., ethanol or another drying solvent.

41. The method of any of claims 29-40, wherein the decellularized tissue is incubated in acetone or another high vapor pressure solvent at about −50° C. to 75° C., −30° C. to 45° C. or −15° C. to 25° C. of any of claims 29-40, wherein the polymer is cross-linked by use of UV cross-linking, chemical cross-linking or a combination thereof.

42. The method of any of the foregoing claims, wherein at least one active is comprised within at least one polymer which is comprised in the graft.

43. The method of claim 42, wherein said active comprises an analgesic, drug, anti-inflammatory, growth factor, hormone, antibiotic, chemotherapeutic, cytokine, anti-rejection agent, or a combination of any of the foregoing.

44. The method of claim 42 or 43, wherein said active is comprised in a hydrogel polymer or other polymer which provides for controlled or sustained release of the active during in vitro or in vivo use.

45. The method of claim 44, wherein different polymers respectively comprising different actives are added which provide for different release rates.

46. The method of any of the foregoing claims, wherein at least one viable cell, a polymer comprising at least one antibiotic, a biodegradable polymer, a non-biodegradable polymer, a polymer capable of cross-linking, chemotactic agent, or a combination thereof is added.

47. A method of grafting to a subject a polymer-impregnated graft, comprising obtaining the polymer-impregnated graft according to any of claims 1-28, and implanting the polymer-permeated graft to a site on the subject; thereby grafting to a subject the polymer-permeated graft.

48. A method of treating a subject afflicted with Pelvic organ prolapse (POP), the method comprising:
obtaining a polymer-impregnated graft according to any of claims 1-28, and
implanting the polymer-permeated graft to in the subject.

49. The method of claim 48, wherein the graft has been repopulated with viable cells and/or metabolically active cells.

50. The method of claim 49, wherein the cells comprise exogenous cells, autologous cells, allogenic cells.

51. The method of any of claims 48-50, wherein the polymer comprises at least one active which is suitable for treating or managing the symptoms of POP.

52. The method of claim 51, wherein said active is comprised in a hydrogel polymer or another polymer that provides for controlled or sustained release of the active.

53. The method or graft of any the previous claims, wherein the polymer comprises alginate, elastin, silk fibroin, gelatin or a combination of 2, 3 or all 4 of the foregoing.

54. The method or graft of any the previous claims wherein the graft comprises at least one active, and the graft provides for sustained, local and/or intermittent release of the active at a desired site, e.g., a tissue or organ in a subject comprising the graft.
